(19) United States
(12) Patent Application Publication
(10) Pub. No.: US 2009/0238724 A1
(43) Pub. Date: Sep. 24, 2009

(54) DISC FOR ANALYZING SAMPLE

(75) Inventors: Tomohiro Yamamoto, Osaka (JP); Nobuhiko Ozaki, Kanagawa (JP)

Correspondence Address: PEARNE & GORDON LLP, 1801 EAST 9TH STREET, SUITE 1200, CLEVELAND, OH, 44114-3108, US

(73) Assignee: MATSUSHITA ELECTRIC INDUSTRIAL CO., LTD., Osaka (JP)

(21) Appl. No.: 12/092,242

(22) PCT Filed: Oct. 31, 2006
(86) PCT No.: PCT/JP2006/321732

§ 371 Date: Apr. 30, 2008

(30) Foreign Application Priority Data
Nov. 2, 2005 (JP) 2005-319716
Dec. 5, 2005 (JP) 2005-350812
Dec. 5, 2005 (JP) 2005-350813

(51) Int. Cl. G01N 33/00; B05D 3/00

(52) U.S. Cl. 422/68.1; 427/2.13

(57) ABSTRACT

It is an object of the present invention to provide a disc for analyzing a fluid sample, which can analyze the fluid sample with accuracy by having the fluid sample react quickly and uniformly with the solid reagent held in the chamber. The disc has a chamber is defined by a gap formed with at least two opening, and defined by a spacer intervening between an upper substrate and a lower substrate, the upper substrate having a convex section, the lower substrate having a concave section, the reagent being attached to the concave section of the lower substrate.

### TECHNICAL FIELD OF THE INVENTION

The present invention relates to a device for detecting chemical reactions of sample and reagent in the field of clinical examination.

### DESCRIPTION OF THE RELATED ART

On account of progress in analysis, diagnosis, and testing technologies in recent years, measurements of various substances have now become possible. Especially in the field of clinical examination, measurement of substances contained in one's body fluid reflected by disease conditions has become possible owing to development of measuring principles based on specific reactions such as biochemical reaction, enzymatic reaction, immune reaction, or the like.

Among other things, ardent attention is paid to “Point of Care Testing”, hereinafter simply referred to as “POCT”, in the field of clinical examination. The primary purpose of the POCT is to achieve a simple and quick testing that reduces time taken after a specimen is collected until the testing result is obtained. Accordingly, the POCT requires a simple testing principle and a testing device which is small in size and excellent in portability and operability.

Recently, the measuring device applied to the POCT has been close to practical use with the establishment of simple measurement principle and following technologies for solidifying an extracted biogenic substance, manufacturing a device, formulation as a system, fine processing, and controlling a microfluid.

As an example applicable to the POCT, a device for performing a quantitative analysis and a qualitative analysis of a fluid sample dropped on a disc is disclosed in patent document 1.

The conventional fluid sample analyzing device disclosed in the patent document 1 can analyze one's blood sample or the like, and is useful in diagnosing diseases. FIG. 16 is a view showing a disc to be used in the conventional fluid sample analyzing device. As shown in FIG. 16, the disc 101 has an inlet port 104 which the fluid sample is introduced through, and a fluid passageway 105 which a reagent 106 has been applied to. When the fluid sample flows through the fluid passageway 105, the reagent 106 applied in the fluid passageway 105 changes in optical features such as for example transmittance and color by reacting chemically with the fluid sample.

The disc 101, into which the fluid sample 900 has been introduced through the inlet port 104, is mounted on the conventional fluid sample analyzing device. The analysis of the fluid sample 900 is then performed by the conventional fluid sample analyzing device.

FIG. 17 is a perspective view showing a conventional fluid sample analyzing device disclosed in the patent document 1. As shown in FIG. 17, the conventional fluid sample analyzing device is similar in construction to so-called “optical disc apparatus”, and comprises a spindle motor 201 having the disc 101 rotated around its rotation axis, an optical pickup 212 for irradiating, with a light beam, the reagent 106 which has reacted chemically with the fluid sample 900 introduced into the disc 101, and a transfer motor 213 for having the optical pickup 212 moved in a radial direction of the disc 101.

When the disc 101 mounted on the conventional fluid sample analyzing device is rotated by the spindle motor 201, the fluid sample 900 flows through the fluid passageway 105 in response to a centrifugal force and reacts chemically with the reagent 106 applied to the fluid passageway 105. After the fluid sample 900 reacts with the reagent 106, the fluid sample 900 or the reagent 106 in the fluid passageway 105 is irradiated with the light beam by the optical pickup 212 under the condition that the disc 101 is being rotated around the rotation center. The detection of the state of chemical reaction of the reagent 106 with the fluid sample 900 and the analysis of the fluid sample 900 is performed on the basis of the reflected light or the transmitted light detected by the optical pickup 212.

In order to obtain a blood plasma component from for example one's blood sample introduced into the above-mentioned disc on the basis of a centrifugal separation method by removing a blood cell component, to have the reagent solved in the blood plasma, and to have the blood plasma react chemically with reagents in a specific sequence, the disc has chambers connected through fluid passageways and reagents applied to the chambers, and has a function having the fluid sample flow and stop in the chambers and the fluid passageways (see for example patent document 2).

The following description is directed to a function having the fluid sample flow or stop in the disc disclosed in the patent document 2, and resulting from a structure disclosed in the patent document 1. FIG. 18 is a diagram explaining technical features of the device disclosed in the patent document 2.

As shown in FIG. 18, the fluid passageway 302 extends from the bottom of an upperstream chamber 301 in view of a direction of a centrifugal force which is used as a reference for explaining a layout, as a channel leading to a position 302a which is above an upper surface of the upperstream chamber 301 in FIG. 18, in a direction opposite to the direction of the centrifugal force which is used as a reference for explaining a layout, further extends in the direction of the centrifugal force, and leads to a downstream chamber 303. Similarly, the fluid passageway 304 extends from the bottom of the downstream chamber 303 in view of a direction of a centrifugal force which is used as a reference for explaining a layout, as a channel leading to a position which is above an upper surface of the downstream chamber 303, in a direction opposite to the direction of the centrifugal force which is used as a reference for explaining a layout, further extends in the direction of the centrifugal force, and leads to a transmitted light measuring chamber 305.

It is important to note that the chamber is deeper than the fluid passageway. This leads to the fact that a section connected to the next chamber can prevent the fluid sample from flowing into the next chamber beyond the section connected to the next chamber even if the fluid sample flows through the fluid passageway to the next chamber in response to the capillary force, and stop the fluid sample just before the next chamber. When the disc for analyzing a sample is rotated around its rotation axis, the centrifugal force acts on the fluid sample stopped just before the next chamber. Then, the fluid sample flows into the next chamber (downstream chamber 303).

It is further important to note that the flow passageway connected to, for example, the upperstream chamber 301 is characterized in that the flow passageway extends, as a channel leading to a position 302a which is above an upper surface of the upperstream chamber 301, in a direction opposite to a direction of a centrifugal force which is used as a reference for explaining a layout, and further extends in the direction of the centrifugal force.

When the centrifugal force acts on the disc, the fluid sample is accumulated in the upperstream chamber 301. When the upperstream chamber 301 is filled with the fluid sample, the fluid sample spilt from the upperstream chamber 301 starts to flow toward the downstream chamber 303 through a U-shaped section corresponding to the position 302a. When the flow passageway 302 is filled with the fluid sample, the fluid sample accumulated in the upperstream chamber 301 starts to be siphoned on the basis of the siphon effect to the downstream chamber 303 through the flow passageway 302. Then, almost all the fluid sample accumulated in the upperstream chamber 301 flows into the downstream chamber 303.

When the centrifugal force is acting on the disc, the fluid sample flows into the fluid passageway 304 from the downstream chamber 303. However, the centrifugal force keeps a balance between the fluid level of the fluid sample flowed into the fluid passageway 304 and the fluid level of the fluid sample accumulated in the downstream chamber 303, and prevents the fluid sample flowed into the fluid passageway 304 from reaching the U-shaped section 302a defined as being above the upper surface of the downstream chamber 303 in view of a direction of a centrifugal force which is used as a reference for explaining a layout. When the centrifugal force is acting on the disc, the fluid sample flowed into the fluid passageway 304 from the downstream chamber 303 can reach a section connected to the next chamber under the condition that the flow passageway 304 is the same in construction as the fluid passageway 302, and characterized as a channel leading to a position which is above an upper surface of the downstream chamber 303.

When the disc is stopped, or when the centrifugal force is removed from the disc, the flow passageway 304 starts to soak up the fluid sample by capillary action. The fluid sample flows into the next chamber through the U-shaped section defined as being above the upper surface of the downstream chamber 303 in view of a direction of a centrifugal force which is used as a reference for explaining a layout, and stops just before flowing into the next chamber, or a transmitted light measuring chamber 305 in FIG. 18. When the centrifugal force acts on the disc again, the fluid sample is siphoned from the downstream chamber 303, and flows into the transmitted light measuring chamber 305. However, the fluid sample flows back from the transmitted light measuring chamber 305 when the centrifugal force is removed from the disc. Therefore, it is necessary to measure the transmitted light from the transmitted light measuring chamber 305 under the condition that the centrifugal force is acting on the disc.

In order to have the fluid sample flow into each chamber effectively, the disc has air openings 306, 307, 308 formed with upper sections of the chambers. Even if the chamber is filled with the fluid sample, the fluid sample cannot reach the upper section. In the disc thus constructed, the reagent can be sufficiently solved in, and react chemically with the fluid sample. The fluid sample can flow through the fluid passageway.

As an example of a disc for analyzing a specific component of a fluid sample, a solution of reagent necessary to analyze the specific component of the fluid sample, and sufficient to react chemically with the fluid sample is introduced into the chamber 301. A reagent layer is formed from the solution of reagent introduced into the chamber 301, and held in the chamber through a step of drying the solution of reagent introduced into the chamber 301.

Patent document 1: International publication No. 00/026677
Patent document 2: Japanese unexamined patent publication No. 2000-580007

### DISCLOSURE OF THE INVENTION

### Problems to be Solved by the Invention

The conventional testing device, however, encounters such a problem that the reagent held as a layer in the chamber 301 tends to be insufficiently solved in the fluid sample introduced into the chamber 301 as a result of the fact that the fluid sample is not agitated with the reagent when the fluid sample is introduced into the chamber 301.

As shown in FIG. 19, the side surface 301a and the lower surface 301b of the chamber 301 are at a right angle or an acute angle with respect each other. When the solid reagent 910 is formed on the lower surface 301b and held in the chamber 301 through a step of drying the solution of reagent introduced into the chamber 301, the solid reagent 910 is nonuniformly deposited while being affected by a surface tension of the solution of reagent or the like, and deposited thickly on the lower surface 301b in the vicinity of the side surface 301a. As a result, the solid reagent 910 deposited on the lower surface 301b tend to be insufficiently solved in the fluid sample introduced into the chamber 301.

In any case, the conventional disc encounters such a problem that the solution of reagent solved in the fluid sample has low concentration on the reagent by reason that the solid reagent 910 is deposited nonuniformly on a section which has a tendency to have the solid reagent react chemically with the fluid sample introduced into the chamber. Consequently, the fluid sample cannot react sufficiently with the reagent held in the chamber, or the fluid sample reacts chemically with the reagent held in the chamber 301 with low reproducibility as a result of the fact that the reagent is solved in the fluid sample with low reproducibility.

It is therefore an object of the present invention to provide a disc for analyzing a sample, which can analyze the fluid sample quickly and accurately by having the reagent deposited uniformly on a section which has a tendency to have the solid reagent react chemically with the fluid sample introduced into the chamber, and by having the fluid sample solve quickly and uniformly, and react quickly and uniformly with the solid reagent held in the chamber.

### Means for Solving the Problems

In order to attain an object of the present invention, a disc for analyzing a fluid sample has surfaces forming a chamber which the fluid sample is introduced into, and a solid reagent held in the chamber, the solid reagent being solved in the fluid sample introduced into the chamber, wherein the disc has at least one of four structural features including: (1) a structural feature characterized in that the surfaces includes a surface, which the solid reagent is attached to, having the shape of concave, (2) a structural feature characterized in that the surfaces includes two surfaces facing each other, one surface, which the solid reagent is attached to, having the shape of concave, the other surface having a section having the shape of convex, (3) a structural feature characterized in that the surfaces includes a reagent attaching surface which the solid reagent is attached to, an opposing surface facing the reagent attaching surface, and a surrounding surface extending from the reagent attaching surface to the opposing surface, the reagent attaching surface having a section inclined with respect to one disc surface and connected to the surrounding surface, the surrounding surface having two sections facing each other, the solid reagent being attached to one section, and not attached to the other section, (4) a structural feature characterized by a columnar section projecting from reagent attaching surface which the solid reagent is attached to, the solid reagent held in the chamber is uniformly deposited on a section which has a tendency to have the solid reagent react chemically with the fluid sample introduced into the chamber. The disc for analyzing a fluid sample thus constructed according to the present invention can analyze the fluid sample quickly and accurately in comparison with the conventional device by having the reagent deposited uniformly on a section which has a tendency to have the solid reagent react chemically with the fluid sample introduced into the chamber, and by having the fluid sample solve quickly and uniformly, and react quickly and uniformly with the solid reagent held in the chamber.

In the disc for analyzing a fluid sample according to the present invention, the chamber is defined by a gap formed with at least two opening, and defined by a spacer intervening between an upper substrate and a lower substrate, the upper substrate having a convex section, the lower substrate having a concave section, the reagent being attached to the concave section of the lower substrate. It is preferable that the convex section of the upper substrate and the concave section of the lower substrate are similar in shape to each other. It is preferable that a distance between the convex section of the upper substrate and the concave section of the lower substrate is substantially fixed in every corner of the convex section and the concave section. It is preferable that the convex section has a curved surface, and the concave section has a curved surface. It is preferable that the disc for analyzing a fluid sample further has two or more chambers connected through one or more fluid passageways, each of the chambers being the same in construction as the chamber.

In other wards, the disc for analyzing a fluid sample according to the present invention may have at least one chamber defined as a gap formed with at least two openings, and a fluid passageway intervening between two chambers, and having one end connected to an opening of one of the chambers and the other end connected to an opening of the other of the chambers, the chambers being held in fluid communication with each other. The upper surface may have the shape of concave, while the lower surface of the chamber may have the shape of convex. The solid reagent may be attached to the lower surface of the chamber. The disc may have a detecting unit for detecting a chemical reaction between the reagent and the fluid sample, and a feeding unit for feeding the fluid sample into the chamber and the fluid passageway.

The disc for analyzing a fluid sample thus constructed according to the present invention can analyze the fluid sample quickly and accurately in comparison with the conventional device by having the reagent deposited uniformly on a section which has a tendency to have the solid reagent react chemically with the fluid sample introduced into the chamber, and by having the fluid sample solve quickly and uniformly, and react quickly and uniformly with the solid reagent held in the chamber.

The disc for analyzing a fluid sample according to the present invention can attain the object of the present invention by having the above-mentioned structural feature (3).

More specifically, the disc for analyzing a fluid sample according to the present invention may have surfaces forming a chamber, and a solid reagent held in the chamber and attached to one of the surfaces, the solid reagent being solved in the fluid sample when the fluid sample is introduced into the chamber, wherein the surfaces includes a reagent attaching surface located on one side of the disc, the solid reagent being attached to the reagent attaching surface, and a surrounding surface extending from the reagent attaching surface, the reagent attaching surface having a section inclined with respect to said one side of the disc, and connected to the surrounding surface, the surrounding surface having two sections facing each other, the solid reagent being attached to one of the sections, and not attached to the other of the sections, and the solid reagent is deposited on the reagent attaching surface through a step of drying a solution of reagent.

When the reagent layer is formed on a surface through a step of drying a solution of reagent in the chamber under the condition that the solution of reagent is pressed in a direction of one surface of the disc, the disc for analyzing a fluid sample thus constructed according to the present invention can analyze the fluid sample quickly and accurately by having the solid reagent deposited uniformly on a section which has a tendency to have the solid reagent react chemically with the fluid sample introduced into the chamber, and react quickly and uniformly with the solid reagent held in the chamber. The disc for analyzing a fluid sample thus constructed according to the present invention can prevent the solution of reagent from reaching a section which the solid reagent is not attached to, and prevent the solid reagent from reaching a section which the solid reagent is not attached to, by reason that, in the disc for analyzing a fluid sample, the reagent attaching surface has a section inclined with respect to said one side of the disc.

In the disc for analyzing a fluid sample according to the present invention, a section to which the solid reagent is attached is connected to a section other than the section inclined with respect to said one side of the disc.

The disc for analyzing a fluid sample thus constructed according to the present invention can prevent the solution of reagent from reaching a section which the solid reagent is not attached to, and prevent the solid reagent from reaching a section which the solid reagent is not attached to.

In a method of producing a disc for analyzing a fluid sample according to the present invention, the disc having surfaces forming a chamber, and a solid reagent held in the chamber and attached to one of the surfaces, the solid reagent being solved in the fluid sample when the fluid sample is introduced into the chamber, wherein the surfaces includes a reagent attaching surface located on one side of the disc, the solid reagent being attached to the reagent attaching surface, and a surrounding surface extending from the reagent attaching surface, the reagent attaching surface having a section inclined with respect to the one side of the disc, and connected to the surrounding surface, the surrounding surface having two sections facing with each other, the solid reagent being attached to one of the sections, and not attached to the other of the sections. The solid reagent may be deposited on the reagent attaching surface through a step of drying a solution of reagent.

The method thus constructed according to the present invention can analyze the fluid sample quickly and accurately in comparison with the conventional device by having the reagent deposited uniformly on a section which has a tendency to have the solid reagent react chemically with the fluid sample introduced into the chamber, and by having the fluid sample solve quickly and uniformly, and react quickly and uniformly with the solid reagent held in the chamber.

The disc for analyzing a fluid sample according to the present invention can attain the object of the present invention by having the above-mentioned structural feature (4).

In other wards, the disc for analyzing a fluid sample according to the present invention may have a chamber formed therein, the disc having a columnar section disposed in the chamber and projected from a surface of the chamber, a solid reagent attached to one surface of the chamber being solved in the fluid sample introduced into the chamber.

The disc for analyzing a fluid sample according to the present invention can analyze the fluid sample quickly and accurately in comparison with the conventional device by having the fluid sample react quickly and uniformly with the solid reagent held in the chamber by reason that, when the fluid sample is introduced into the chamber, the fluid sample can be agitated by the columnar section. The disc for analyzing a fluid sample according to the present invention can have the reagent deposited uniformly on a section which has a tendency to have the solid reagent react chemically with the fluid sample introduced into the chamber, by reason that the solid reagent is thickly deposited in the vicinity of the columnar section when the solid reagent is held in the chamber through a step of drying the solution of reagent in the chamber, and can analyze the fluid sample quickly and accurately in comparison with the conventional device.

The disc for analyzing a fluid sample according to the present invention may have two or more columnar sections disposed at intersecting points of an equilateral triangle grid.

The disc for analyzing a fluid sample thus constructed according to the present invention can have the reagent deposited uniformly on a section which has a tendency to have the solid reagent react chemically with the fluid sample introduced into the chamber, by reason that the columnar sections are located in regular intervals.

The disc for analyzing a fluid sample according to the present invention has two or more columnar sections disposed at intersecting points of an orthogonal grid.

The disc for analyzing a fluid sample thus constructed according to the present invention can have the reagent deposited uniformly in comparison with a disc having two or more columnar sections disposed at intersecting points of an equilateral triangle grid, by reason that the columnar sections are located in regular intervals.

In the disc for analyzing a fluid sample according to the present invention, the columnar section extends from one of surfaces facing with each other, and is connected to the other of the surfaces.

The disc for analyzing a fluid sample thus constructed according to the present invention can strongly agitate the fluid sample in the chamber when the fluid sample flows into the chamber, in comparison with reason that the columnar section extending from one of the surfaces toward the other of the surfaces.

In the disc for analyzing a fluid sample according to the present invention, the columnar section extends from one of the surfaces toward the other of the surfaces.

The disc for analyzing a fluid sample thus constructed according to the present invention can allow the fluid sample to quickly flow through a gap between the columnar section extending from one of the surfaces and the other of the surfaces.

In the method of producing a disc for analyzing a fluid sample according to the present invention, the disc has surfaces forming a chamber which the fluid sample is introduced into, and a columnar section disposed in the chamber and projected from a surface which a solid reagent is attached to, the solid reagent being solved in the fluid sample introduced into the chamber. The method may have a step of having the solid reagent attached to the surface of the chamber by drying a solution of reagent in the chamber.

The disc produced on the basis of the method according to the present invention can have the solid reagent react with the fluid sample quickly and uniformly, and analyze the fluid sample quickly and accurately by having a chamber reduced in capacity, by having a solid reagent solved quickly in the fluid sample introduced into the chamber, and having the solved reagent react uniformly with the fluid sample, by reason that, as a result of the fact that the solid reagent is thickly deposited in the vicinity of a columnar section projected from one of the surfaces, the disc has a solid reagent deposited uniformly on a section which has a tendency to have the solid reagent react chemically with the fluid sample introduced into the chamber. Furthermore, the disc produced on the basis of the method according to the present invention can analyze the fluid sample quickly and accurately by reason that, when the fluid sample is introduced into the chamber, the fluid sample and the solid reagent are uniformly agitated by the columnar section.

### ADVANTAGEOUS EFFECT OF THE INVENTION

The disc for analyzing a sample according to the present invention can detect a specific component from a small amount of fluid sample with accuracy by having a chamber reduced in capacity, having a solid reagent quickly solved in the fluid sample introduced into the chamber, and having the solved reagent uniformly react chemically with the fluid sample, by reason that the disc has a solid reagent deposited uniformly on a section which has a tendency to have the solid reagent react chemically with the fluid sample introduced into the chamber.

### BRIEF DESCRIPTION OF THE DRAWINGS

FIG. 1 is a schematic top view partially showing a disc for analyzing a sample according to one embodiment of the present invention.

FIG. 2 is a sectional view taken along a line a-a′ of FIG. 1.

FIG. 3 is a plan view showing a disc for analyzing a sample according to one embodiment of the present invention.

FIG. 4 is a plan view partially showing the disc for analyzing a sample according to one embodiment of the present invention.

FIG. 5(a) is a plan view showing a reagent holding chamber and its surroundings of the disc for analyzing a sample according to the embodiment of the present invention. FIG. 5(b) is a sectional view taken along a line A-A′ of FIG. 5(a).

FIG. 6(a) is a graph showing a change of absorbance with respect to a total cholesterol value of a blood plasma component extracted from one's blood sample under the condition that the reagent holding chamber has a shape shown in FIG. 5. FIG. 6(b) is a graph showing a change of absorbance with respect to a total cholesterol value of a blood plasma component extracted from one's blood sample under the condition that the reagent holding chamber is the same in shape as that of the conventional disc. FIG. 6(c) is a graph showing a change of absorbance with respect to a total cholesterol value of a blood plasma component extracted from one's blood sample under the condition that the reagent holding chamber has a shape the same in shape as the reagent holding chamber shown in FIG. 2.

FIG. 7 is a sectional view showing a reagent holding chamber and its surroundings of the disc for analyzing a sample according to one embodiment of the present invention.

FIG. 8 is a plan view showing a reagent holding chamber different from the reagent holding chamber shown in FIG. 5 and its surroundings of the disc for analyzing a sample according to the embodiment of the present invention.

FIG. 9(a) is a plan view showing a reagent holding chamber different from each of the reagent holding chambers shown in FIGS. 5 and 8 and its surroundings of the disc for analyzing a sample according to the embodiment of the present invention. FIG. 9(b) is a sectional view taken along a line B-B′ of FIG. 9(a).

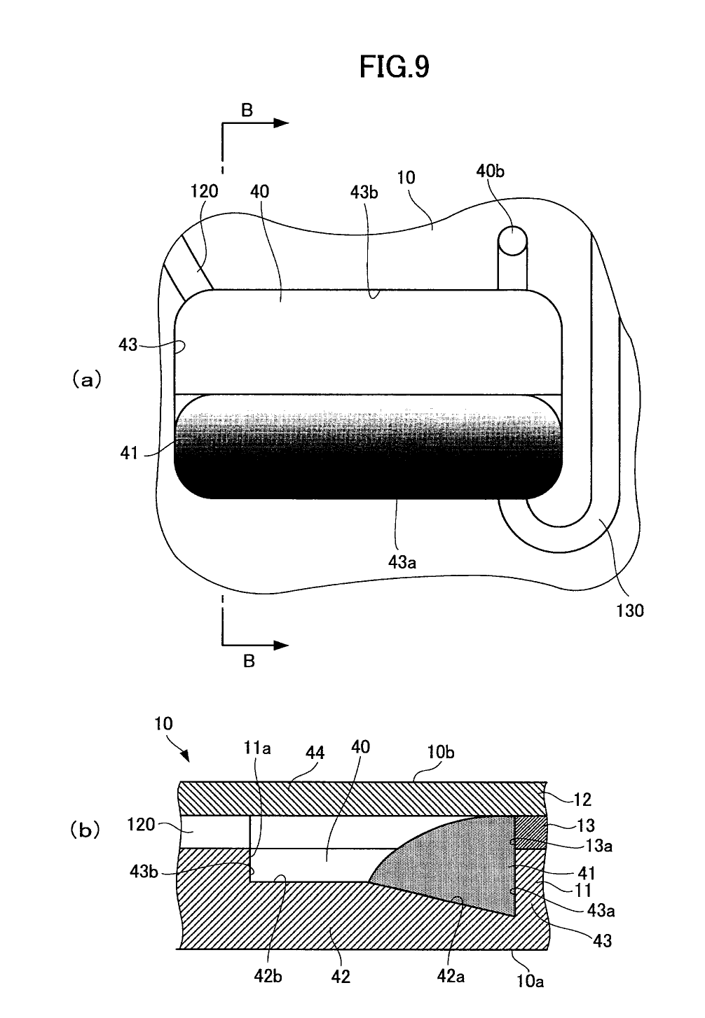

FIG. 10 is a plan view showing a disc for analyzing a sample according to another embodiment of the present invention.

FIG. 11 is a plan view partially showing the disc for analyzing a sample according to another embodiment of the present invention.

FIG. 12(a) is a plan view showing a reagent holding chamber and its surroundings of the disc shown in FIG. 10. FIG. 12(b) is a sectional view showing a reagent holding chamber and its surroundings of the disc shown in FIG. 10.

FIG. 13 is a plan view showing a reagent holding chamber different from the reagent holding chamber shown in FIG. 12 and its surroundings of a disc for analyzing a sample according to the embodiment of the present invention.

FIG. 14 is a plan view showing a reagent holding chamber different from each of the reagent holding chambers shown in FIGS. 12 and 13 and its surroundings of the disc for analyzing a sample according to the embodiment of the present invention.

FIG. 15 is a plan view showing a reagent holding chamber different from each of the reagent holding chambers shown in FIGS. 12, 13, and 14 and its surroundings of the disc for analyzing a sample according to the embodiment of the present invention.

FIG. 16 is a view showing a disc to be used with a conventional fluid sample analyzing device.

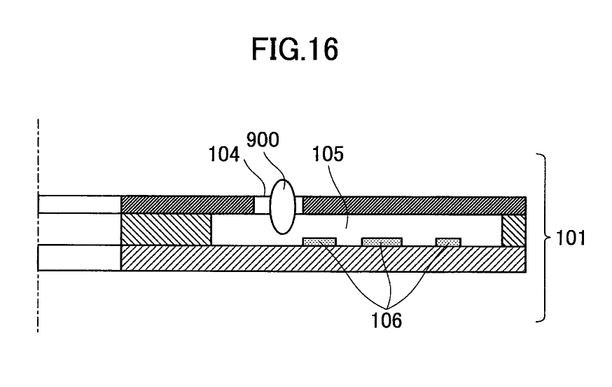

FIG. 17 is a view showing a conventional fluid sample analyzing device.

FIG. 18 is a schematic diagram explaining a fluid sample transferring mechanism of a disc for analyzing a sample according to the present invention and the conventional disc.

FIG. 19 is a sectional view showing a chamber and its surroundings of the conventional disc for analyzing a sample.

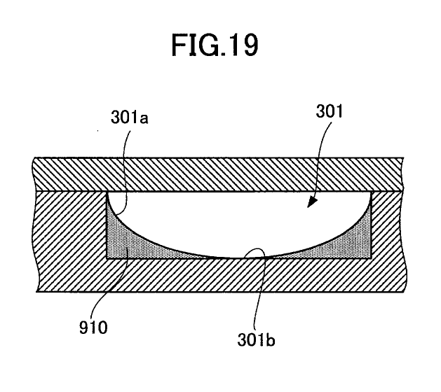

### EXPLANATION OF THE REFERENCE NUMERALS

1, 2: opening section
3: chamber
4: convex section
5a: concave section
5b: reagent layer
6: upper substrate
7: spacer
8: lower substrate
101: disc for analyzing a fluid sample
104: inlet port through which fluid sample is introduced
105: fluid passageway
106: reagent
201: spindle motor
900: sample
212: optical pickup
213: transfer motor
302, 304: fluid passageway
301, 303, 305: chamber
306, 307, 308: air opening
10: disc for analyzing a sample
10a: surface (one surface of the disc)
10b: surface
40: reagent holding chamber
41: ChE layer (solid reagent)
42: surface (surface to which a reagent is attached)
42a: slanted section
42b: horizontal section (other than a slanted section)
42c: columnar section
43: surface (surrounding surface)
43a: a section to which a reagent is attached
43b: a section to which a reagent is not attached
44: surface
48: orthogonal grid
49: equilateral triangular grid
50: reagent holding chamber
51: ChDH layer (solid reagent)
52: columnar section
60: reagent holding chamber
61: WST-9 layer (solid reagent)
62: columnar section

### DESCRIPTION OF THE PREFERRED EMBODIMENTS

The disc for analyzing a sample according to the first to third embodiments of the present invention will be described hereinafter with reference to the accompanying drawings.

### First Embodiment

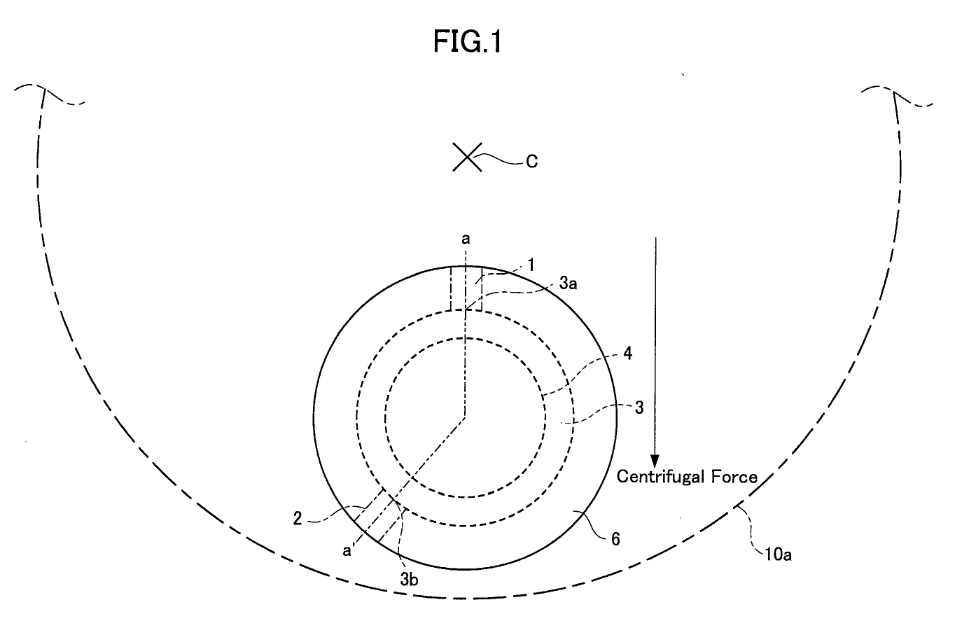

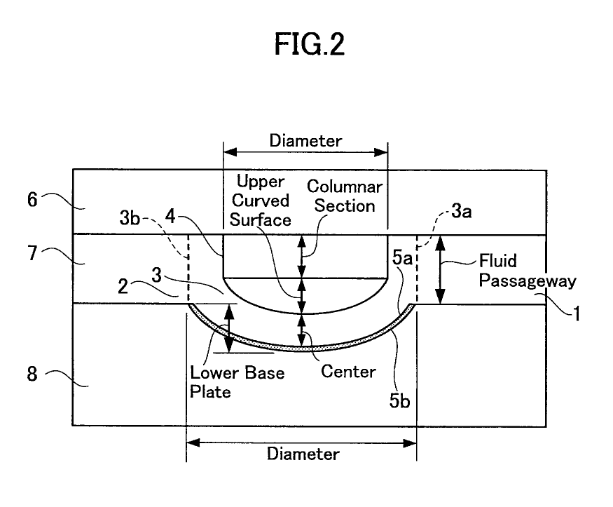

FIG. 1 is a schematic top view partially showing a disc for analyzing a sample according to one embodiment of the present invention. In FIG. 1, the disc for analyzing a sample is illustrated with an alternate long and short dash line. The disc has a rotation center illustrated with a reference character “C”. FIG. 2 is a sectional view taken along a line a-a′ of FIG. 1.

As shown in FIGS. 1 and 2, the disc for analyzing a sample according to the present invention has a chamber 3 formed as a gap, defined by a spacer 7 intervening between a upper substrate 6 and a lower substrate 8. formed with at least two opening sections 1 and 2, and The upper substrate 6 has a convex section 4 formed as an upper surface of the chamber 3, while the lower substrate 8 has a concave section 5a formed as a lower surface of the chamber 3, a reagent layer 5b being deposited on the concave section 5a.

In this embodiment, the chamber 3 is formed by the upper substrate 6 and the lower substrate 8. However, the chamber 3 may be formed on a disc by another member. A disc provided with a chamber may be constituted by the upper substrate 6 and the lower substrate 8. The disc constituted by the upper substrate 6 and the lower substrate 8 may have a chamber formed therein. In this embodiment, the upper substrate 6 and the lower substrate 8 collectively forms a chamber 3 illustrated with an alternate long and short dash line 10a as shown in FIGS. 1 and 2.

In the disc for analyzing a sample, the chamber 3 is surrounded by boundary lines 3a and 3b. The sample (such as fluid) flows into the chamber 3 through passageway (not shown) connected to an opening section 1 close to the rotation center C, and flows out from the chamber 3 through the opening section 2.

In order to provide an easy-to-understand explanation about the chamber 3, the chamber 3 has the shape of circle as shown in FIG. 1. However, the chamber 3 may be arbitrarily defined in shape under the condition that the concave section 5a is formed on a bottom surface (i.e. lower substrate 8) in the chamber 3. A projection formed as a combination of an upper curved surface similar to the concave section 5a and a cylinder section, i.e., the convex section 4 and a cylinder section, the shape of a planar projection of the upper curved surface being defined as a bottom surface of the cylinder section. As shown in FIG. 2, it is preferable that the convex section 4 be surrounded in size by the concave section 5a.

The convex section 4 of the upper substrate 6 and the concave section 5a of the lower substrate 8 are similar in shape to each other. A distance between the convex section 4 of the upper substrate 6 and the concave section 5a of the lower substrate 8 is substantially fixed in every corner of the convex section 4 and the concave section 5a.

The disc for analyzing a sample according to the present invention has a reagent layer 5b formed on the lower surface of the chamber 3, or the concave section 5a. The reagent layer 5b may be constituted by a solid reagent, or may be constituted by an undried reagent such as a semisolid or a gelled reagent.

As shown in FIG. 2, the disc for analyzing a sample according to the present invention is constituted by elements stacked in layers in order of the upper substrate 6, the spacer 7, and the lower substrate 8. The thickness of the spacer 7 is the same as the height of the opening sections 1 and 2 and fluid passageways (not shown).

The spacer 7 is formed with fluid passageways connected to the respective opening sections 1 and 2 of the chamber 3. As part of the upper substrate 6, the ceiling of the opening section 1 and its surrounding section is flush with that of the fluid passageway connected to the opening section 1. As part of the upper substrate 8, the concave section 5a is constituted by the slanted lower surface of the chamber 3.

When the fluid sample flows toward the chamber 3, the fluid sample flows to the opening section 1 through the fluid passageway by capillary action, and stops just before the concave section 5a, i.e., just before the boundary line 3a. When the disc for analyzing a sample is rotated again around its rotation axis, the fluid sample starts to flow into the chamber 3 through the opening 2 connected to the fluid passageway in response to the centrifugal force.

The disc for analyzing a sample according to the present invention may have two or more chambers each of which is the same in construction as the above-mentioned chamber 3. In this case, the sample flows from each chamber to the following chamber (not shown) through a fluid passageway connected to the opening 2.

The reagent layer 5b is held in the chamber 3 under the condition that the reagent layer 5b is attached to all over the concave section 5a. As mentioned above, the ceiling of the chamber 3, i.e., the convex section 4 of the chamber 3 is similar in shape as the concave section 5a of the chamber 3 in the vicinity of the center of the chamber 3.

On the assumption that the upper substrate 6 does not have a convex section 4, the distance from the ceiling surface of the chamber 3 to the bottom surface of the chamber 3 is changed with distance from the center of the chamber 3, by reason that the bottom surface of the chamber 3 is curved. On the other hand, the reagent layer 5b is formed on the concave section 5a, and substantially even in thickness in every corner of the concave section 5a. Therefore, the reagent even can be efficiently solved in the fluid sample flowed into the chamber 3 by reason that the reagent layer 5b is formed on the concave section 5a, and substantially even in thickness in every corner of the concave section 5a. However, it is preferable that the upper substrate 6 have convex section 4. The reason simply comes from the fact that the concentration of the reagent in the fluid sample is even in every corner of the chamber 3 when the reagent layer is solved in the fluid sample flowed into the chamber 3. The surface of the reagent layer 5b to the volume of the fluid sample flowed into the chamber 3 is relatively large in comparison with the conventional disc by reason that the chamber 3 has a reduced capacity resulting from the convex section 4 provided in the chamber 3. The depth H of the concave section 5a of chamber 3 formed on the lower substitute 8, the height H of the ceiling of the fluid passageway, the height H of the ceiling of the chamber 3 at the center of the chamber 3, and each dimension (shape of curved surface and height H of columnar section) the convex section 4 of the upper substrate 6 are as follows.

Firstly, it is imperative that the fluid passageway is small in height enough to soak up a fluid sample by capillary action, and generally ranges from 50 μm to 300 μm in height. On the other hand, there are not any limits in depth and size of the concave section 5a of the lower substrate 8. However, a step is provided at the boundary 3a of the opening section 1, and inclined and functioning as a portion having the fluid sample stop flowing into the chamber when the fluid sample flows toward the chamber through the fluid passageway in response to the capillary force. When the reagent layer is formed through a step of drying the solution of reagent in the chamber, the solution of reagent is prevented from flowing from the section. Therefore, it is preferable that the convex section 4 be surrounded in size by the concave section 5a.

In general, the distance between the convex section 4 and the concave section 5a may be 200 to 600 μm. However, it is not necessary that the distance between the convex section 4 and the concave section 5a is within 200 to 600 μm.

It is preferable that the distance (H (at the center)) between the convex section 4 and the concave section 5a be generally the same as the height (H (in the fluid passageway)) of a ceiling from a bottom surface under the condition that the spacer 7 is adhered well to the upper substrate 6 and the lower substrate 8.

The height of the convex section 4 (total of the cylindrical section and upper curved surface) exceed the distance from the bottom to the ceiling of the chamber. The convex section 4 may be partially received in the concave section 5a.

The reagent layer 5a is formed through a step of drying the solution of the reagent dropped on a concave section 5b of the lower substrate 8. The concentration of the solution of the reagent and the volume of the solution of the reagent is determined on the basis of the ratio of the volume of the concave section to the volume of the chamber 3. The shape of the convex section 4 of the upper substrate 6 is determined under the condition that the convex section 4 of the upper substrate 6 does not contact with the opening 1 of the chamber 3. The reagent layer may be formed through a step of drying an aqueous solution, pH buffer, other solution of the reagent dropped on a concave section 5b. The disc for analyzing a fluid sample according to the present invention may have two or more chambers. It is preferable not to mix three reagents to be used for determining for example a total cholesterol value by reason that the reagents to be used for determining for example a total cholesterol value are denatured in a chamber as a result of the fact that the reagents interact with each other.

The first embodiment of the disc for analyzing a sample according to the present invention will be described hereinafter in detail with examples such as fluid samples to be analyzed and materials of members of the disc. However, the disc for analyzing a sample according to the present invention is not limited by the examples which will be described hereinafter.

### (Example of First Embodiment)

In order to determine a total cholesterol value, we have produced a disc for from a blood plasma component extracted from one's blood sample, the disc having a chamber 3 shown in FIGS. 1 and 2. We have prepared an upper substrate 6 made from polycarbonate, a lower substrate 8 made from polycarbonate, and a spacer 7 made from polyethylene terephthalate, the spacer 7 having a thickness of 100 μm, the spacer 7 having two-sided adhesive tapes attached thereon, the spacer 7 being adhered well to the upper substrate 6 and the lower substrate 8. The lower substrate 8 is formed by a molding machine, and has a concave section 5b. On the other hand, the upper substrate 6 is formed by the molding machine, and has a convex section 4.

The concave section 5b has a curved surface and the shape of circle having a diameter of 2 mm, a depth of 0.4 mm at a center of the circle, and a capacity of approximately 2.78 μl. The spacer 7 has a groove formed therein, the groove having a width of 0.5 mm, and being located as a fluid passageway to be connected to the chamber 3.

The upper substrate 6 has a section formed with a projection, the section having a cylindrical section having a height of 0.1 mm having an upper circular surface having a diameter of 1.7 mm, and a lower circular surface having a diameter of 1.7 mm.

Under the condition that the convex section 4 is 0.4 mm in overall height, the distance (H (at the center)) from the top of the convex section 4 to the concave section 5a is 0.1 mm, and substantially fixed within an area where the convex section 4 and the concave section 5a are in face-to-face relationship with each other. In this case, the chamber 3 is 1.10 μl in capacity.

The disc for analyzing a sample according to the present invention is produced through steps of preparing an upper substrate 6, a lower substrate 8, and a spacer 7 by cutting out chambers and fluid passageways from the upper substrate 6, the lower substrate 8, and the spacer 7 on the basis of their shapes, the spacer 7 having surfaces each having a two-sided adhesive tape attached thereon, and having the spacer 7 adhered well to the upper substrate 6, the lower substrate 8 after having a reagent layer 5b attached to the concave section 5a.

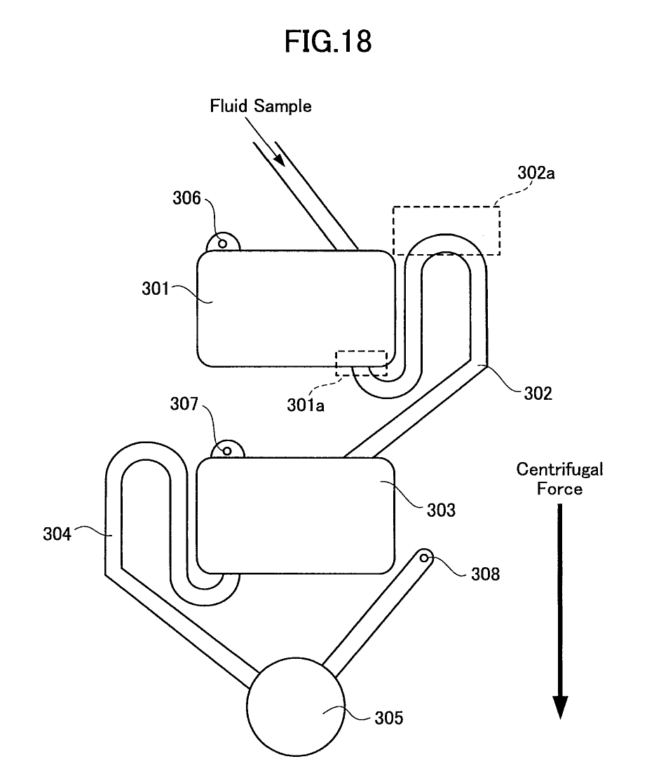

The reagent layer 5b is formed through steps of preparing a water solution or a pH buffer solution in which one or more reagents needed to react chemically with the fluid sample are solved, having the solution introduced into the chamber 3, and drying the solution introduced into the chamber 3. For example, reagents needed to determine a total cholesterol value are denaturalized by interactions between/among the reagents under the condition that the reagents are mixed and held in a chamber in the form of a layer. As a result, it is difficult to obtain desired chemical reactions from the denaturalized reagents. Therefore, it is preferable that the reagents be held in respective chambers. In this case, the fluid passageway 302, connected to a section 301a defined as part of a bottom of an upperstream chamber 301 on the basis of a direction of a centrifugal force which is used as a reference for explaining a layout, extends, as a channel leading to a position 302a which is above an upper surface of the upperstream chamber 301 in FIG. 18, in a direction opposite to the direction of the centrifugal force which is used as a reference for explaining a layout, further extends in the direction of the centrifugal force, and leads to a downstream chamber 303. Similarly, the fluid passageway 304, connected to a section defined as part of a bottom of the downstream chamber 303 on the basis of the direction of the centrifugal force which is used as a reference for explaining a layout, extends, as a channel leading to a position which is above an upper surface of the downstream chamber 303, in a direction opposite to the direction of the centrifugal force which is used as a reference for explaining a layout, further extends in the direction of the centrifugal force, and leads to a transmitted light measuring chamber 305.

It is further important to note that the flow passageway connected to, for example, the upperstream chamber 301 is characterized in that the flow passageway extends, as a channel leading to a position 302a which is above an upper surface of the upperstream chamber 301, in a direction opposite to a direction of a centrifugal force which is used as a reference for explaining a layout, and further extends in the direction of the centrifugal force.

When the centrifugal force acts on the disc, the fluid sample is accumulated in the upperstream chamber 301. When the upperstream chamber 301 is filled with the fluid sample, the fluid sample spilt from the upperstream chamber 301 starts to flow toward the downstream chamber 303 through a U-shaped section corresponding to the position 302a. When the flow passageway 302 is filled with the fluid sample, the fluid sample accumulated in the upperstream chamber 301 starts to be siphoned on the basis of the siphon effect to the downstream chamber 303 through the flow passageway 302. Then, almost all the fluid sample accumulated in the upperstream chamber 301 flows into the downstream chamber 303.

When the centrifugal force is acting on the disc, the fluid sample flows into the fluid passageway 304 from the downstream chamber 303. However, the centrifugal force keeps a balance between the fluid level of the fluid sample flowed into the fluid passageway 304 and the fluid level of the fluid sample held in the downstream chamber 303, and prevents the fluid sample flowed into the fluid passageway 304 from reaching the U-shaped section 302a defined as being above the upper surface of the downstream chamber 303 in view of a direction of a centrifugal force which is used as a reference for explaining a layout. When the centrifugal force is acting on the disc, the fluid sample flowed into the fluid passageway 304 from the downstream chamber 303 can reach a section connected to the next chamber under the condition that the flow passageway 304 is the same in construction as the fluid passageway 302, and characterized as a channel leading to a position which is above an upper surface of the downstream chamber 303.

When the disc is stopped, or when the centrifugal force is removed from the disc, the flow passageway 304 starts to soak up the fluid sample by capillary action. The fluid sample flows into the next chamber through the U-shaped section defined as being above the upper surface of the downstream chamber 303 in view of a direction of a centrifugal force which is used as a reference for explaining a layout, and stops just before flowing into the next chamber, or a transmitted light measuring chamber 305 in FIG. 18. When the centrifugal force acts on the disc again, the fluid sample is siphoned from the downstream chamber 303, and flows into the transmitted light measuring chamber 305. However, the fluid sample flows back from the transmitted light measuring chamber 305 when the centrifugal force is removed from the disc. Therefore, it is necessary to measure the transmitted light from the transmitted light measuring chamber 305 under the condition that the centrifugal force is acting on the disc.

In order to have the fluid sample flow into each chamber effectively, the disc has air openings 306, 307, 308 formed with upper sections of the chambers. Even if the chamber is filled with the fluid sample, the fluid sample cannot reach the upper section. In the disc thus constructed, the reagent can be sufficiently solved in, and react chemically with the fluid sample. The fluid sample can flow through the fluid passageway.

As an example of a disc for analyzing a specific component of a fluid sample, a solution of reagent necessary to analyze the specific component of the fluid sample, and sufficient to react chemically with the fluid sample is introduced into the chamber 301. A reagent layer is formed from the solution of reagent introduced into the chamber 301, and held in the chamber through a step of drying the solution of reagent introduced into the chamber 301.

The detection of a chemical change resulting from a chemical reaction of reagent with cholesterol in one's fluid sample is performed through a step of detecting the change of absorbance of pigment contained in the reagent layer 5b in specific wavelength. Therefore, a section defined as a ceiling of the measuring chamber 305 and a section defined as a bottom of the measuring chamber 305 have respective flat surfaces. Those sections corresponding to the measuring chamber 305 are transmissive within an optical range and the like including the specific wavelength.

Optimally, the light measuring chamber corresponding to reagents which will be described hereinafter has a depth of 200 μm. In general, it is necessary to determine the depth of the measuring chamber, as an optical path, on the basis of attenuation or intensity of light from the measuring chamber to determine the concentration of a specific component contained in the fluid sample in the measuring chamber.

The chamber, in which the reagent layer 5b is held, has a capacity of about 1.1 μl. The measuring chamber has a diameter of 2 mm, and a depth of 200 μm. In this case, the measuring chamber has a capacity of about 0.6 μl. Even if the fluid sample is partially left in the fluid passageway when the fluid sample flows through the fluid passageway to the measuring chamber, the quantity of the fluid sample exceeds a requisite minimum level in the measuring chamber.

The chemical reaction mechanism needed to determine a total cholesterol value is as follows.

E-Chol→Chol(enzyme: ChE) Chemical reaction formula (1)

Chol+NAD→Cholesten+NADH(enzyme: ChDH) Chemical reaction formula (2)

NADH+WST-9→NAD+Formazan (enzyme: DI) Chemical reaction formula (3)

The total cholesterol value is calculated from the measured change of absorbance at a wavelength of 650 nm of the transmitted light from the measuring chamber, resulting from Formazan produced in process of the chemical reaction expressed by the chemical reaction formula (3). Here, “E-Chol” appearing in Chemical reaction formulas (1) to (3) expresses cholesterol ester. Almost all the cholesterol component extracted from the blood plasma is occupied by esterified cholesterol. “Chol” appearing in Chemical reaction formula (2) expresses cholesterol. “ChE” expresses cholesterol esterase (EC3.1.1.13) functioning as an enzyme to catalyze the chemical change from E-Chol to Chol.

“ChDH” expresses cholesterol dehydrogenase (bought from Amano Enzyme Inc.). “NAD” expresses nicotine adenine dinucleotide functioning as coenzyme of ChDH. “NADH” expresses reduced NAD. “WST-9” stands for “water-soluble tetrazolium-9” and expresses a kind of water-soluble tetrazolium which available from Dojindo Laboratories. “DI” expresses diaphorase (EC1.6.99.2) functioning as enzyme to catalyze the chemical change from NADH to NAD (oxidation reaction) and vice versa (reduction reaction).

In order to bring about the chemical reactions expressed by the chemical reaction formulas (1) to (3), the following reagent layers 5b are respectively held in the reagent holding chambers 301, 303, and 305. ChE layer including ChE is held as the first reagent layer. ChDH layer including ChDH is hold as the second reagent layer. WST-9 layer including WST-9 is hold as the third reagent layer.

The ChE layer is formed in the chamber 3 through steps of placing an aqueous solution on the concave section 5a of the lower substrate 8. The aqueous solution consists of ChE, n-octyl-β-D-thioglucoside and sodium cholate which function as a surfactant to increase a catalytic activity of ChE, Tris-hydrochloride which functions as pH buffering agent to adjust pH value of the fluid sample in a chemical reaction, and DI. 2.78 μl of aqueous solution is 1.10/2.78 times larger in concentration than a solution which is necessary to react chemically with the fluid sample in the chamber 3.

The ChDH layer is formed from solution of DhDH and DI through a step of drying the solution of DhDH and DI in the chamber.

The pH buffering agent is essential to adjust the hydrogen-ion exponent of the solution to an optimum hydrogen-ion exponent, or to alkalize the fluid sample. However the stability of ChDH is reduced in the alkaline solution. Therefore, the ChE layer 41 and the ChDH layer 51 are respectively held in the reagent holding chambers 40 and 50 in the disc 10 for determining a total cholesterol value. The pH buffering agent is contained in the ChE layer 41. As ChE, enzyme may be selected from among marketed products on the basis of the stability and the reactive property of the enzyme.

Considering chemical reactions of solid reagents with a fluid sample and time required for determination of a total cholesterol value or the like, it is preferable to reduce the numbers of chambers in which the solid reagents are respectively held. In the disc 10 for determining a total cholesterol value, the ChE layer 41, the ChDH layer 51, and the WST-9 layer 61 are respectively held in the reagent holding chambers 40, 50, and 60 by reason that WST-9 has a tendency to inhibit a catalytic activity of ChDH. Therefore, chemical reactions represented by the chemical reactions (1) and (2) are respectively yielded in chambers in which WST-9 is not held. In order to yielded color reaction under the condition that WST-9 is finally solved in the next chamber, it is important that ChE layer, ChDH layer, and WST-9 layer are respectively held in chambers in order of ChE layer, ChDH layer, and WST-9.

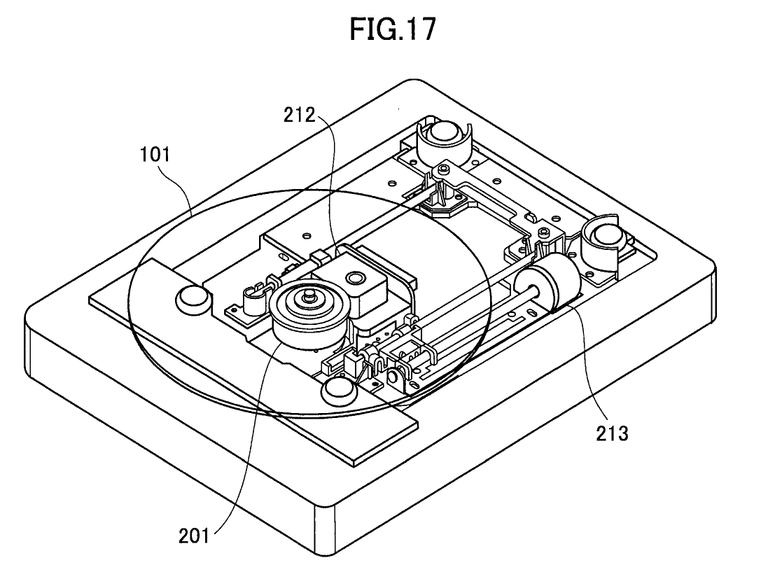

In this embodiment, the blood sample is firstly put on the disc for analyzing a sample, which has been produced on the basis of the above-mentioned method, and which has a reagent layer 5b. The chemical change resulting from a chemical reaction between reagent and cholesterol contained in the blood sample is then measured from the transmitted light from the disc for analyzing a sample is then measured by an analyzing device shown in FIG. 17.

In this embodiment, the disc for determining a fluid sample is exemplified by a disc for determining the total cholesterol value of the blood plasma. The total cholesterol value of the blood plasma is determined from the change of absorbance of WST-9. The reagent layer may contain potassium ferricyanide in place of WST-9, the potassium ferricyanide contained in the reagent layer is reduced to ferricyanide ion in aqueous solution. The disc may have at least two electrodes provided in the measuring chamber. One functions as a counter electrode, while the other functions as a working electrode.

In this embodiment, the disc for determining a fluid sample is exemplified by a disc for determining the total cholesterol value of the blood plasma. The total cholesterol value of the blood plasma is determined from the change of absorbance of WST-9. The reagent layer may contain potassium ferricyanide in place of WST-9, the potassium ferricyanide contained in the reagent layer is reduced to ferricyanide ion in aqueous solution. The disc may have at least two electrodes provided in the measuring chamber. One functions as a counter electrode, while the other functions as a working electrode.

The disc for analyzing a sample according to the present invention is not limited by the analysis of total cholesterol value of the blood plasma explained in this embodiment, and may be applied to an analysis of another component of any sample through an optical change or an electrochemical change of the component.

In this embodiment, a section to which the reagent is attached has the shape of concave. The section projected from a surface facing a reagent attaching surface has the shape of convex, and is surrounded by the reagent attaching surface. However, the section may be arbitrarily defined in shape so as to equalize in thickness the reagent layer formed on a section which has a tendency to have the reagent react chemically with the fluid sample in the chamber. For example, a section to which the reagent is attached has the shape of concave, while a surface facing the reagent attaching surface is flat.

In order to equalize the thickness of the reagent layer formed on a section which has a tendency to have the reagent react chemically with the fluid sample in the chamber, the reagent layer may be formed on only a section which has a tendency to have the reagent react chemically with the fluid sample in the chamber. The following description is directed to the case that the reagent layer is formed on only a section which has a tendency to have the reagent react chemically with the fluid sample in the chamber.

### Second Embodiment

The second embodiment of the disc for analyzing a sample according to the present invention will be then described hereinafter with reference to accompanying drawings.

In order to provide a simple explanation about the disc according to the present invention, the following description is directed to the case that the disc of the present invention is used as a disc for determining a total cholesterol value from a blood plasma component extracted from one's blood sample.

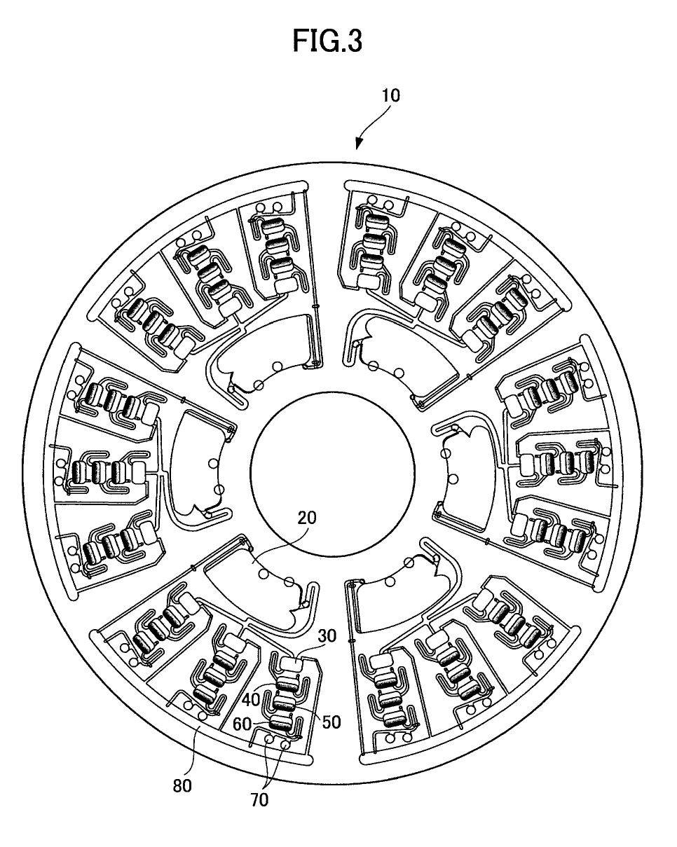

The disc 10 shown in FIG. 3 as a disc for determining a total cholesterol value from a blood plasma component extracted as a fluid sample is mounted on a device shown in FIG. 17.

The disc 10 has a mechanism the same as a mechanism described in the foregoing example.

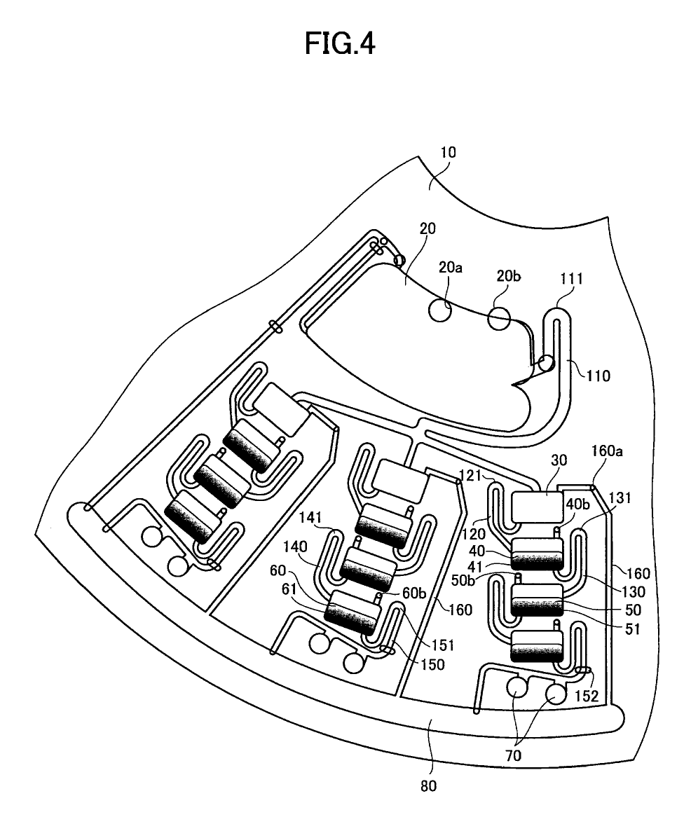

As shown in FIGS. 3 and 4, the disc 10 has a blood cell component separating chamber 20 for removing, on the basis of a centrifugal separation method, a blood cell component from one's blood sample to extract a blood plasma component from the blood sample, a quantity determining chamber 30 for determining and delivering a predetermined quantity of blood plasma component, a reagent holding chamber 40 having a ChE layer 41 deposited as a solid reagent containing ChE, a reagent holding chamber 50 having a ChDH layer 51 deposited as a solid reagent containing ChDH, a reagent holding chamber 60 having a WST-9 layer 61 deposited as a solid reagent containing WST-9, a measuring chamber 70 for measuring an absorbance of Formazan at a wavelength of 650 nm, and a waste fluid chamber 80 having a waste fluid stored therein.

The disc 10 has six blood cell component separating chambers 20 equiangularly disposed on a circumferential line of a circle, quantity determining chambers 30 divided into groups related to the respective blood cell component separating chambers 20, each group being constituted by three quantity determining chambers 30, reagent holding chambers 40 related to the respective quantity determining chambers 30, reagent holding chambers 50 related to the respective reagent holding chambers 40, reagent holding chambers 60 related to the respective reagent holding chambers 50, measuring chambers 70 divided into groups related to the respective reagent holding chambers 60, each group being constituted by two measuring chambers 70, and waste fluid chambers 80 related to the respective blood cell component separating chambers 20.

The disc 10 has inlet ports 20a through which the blood sample is introduced into the blood cell component separating chambers 20, and air openings 20b, 40b, 50b and 60b respectively located at specific positions, the fluid flows smoothly in the disc 10 with air introduced into fluid passageways or chambers through the air openings 20b, 40b, 50b and 60b. When the disc 10 for determining a total cholesterol value is mounted on and being driven by an analyzing device (see FIG. 17), the fluid cannot leak from the disc 10 through the inlet port 20a and the air openings 20b, 40b, 50b and 60b respectively located at specific positions.

Each of the reagent holding chambers 40, 50, and 60 has the shape of a rectangle which measures 2.0 mm by 5.0 mm in a sectional view taken by a plane perpendicular to a thickness direction of the disc 10 for determining a total cholesterol value. Each of the reagent holding chambers 40, 50, and 60 is disposed under the condition that the long side of the rectangle, i.e., the side of 5.0 mm is substantially perpendicular to a radius direction of the disc 10 for determining a total cholesterol value. The depth of the reagent holding chambers 40, 50, and 60 ranges from 200 μm to 400 μm in the thickness direction.

The quantity determining chamber 30 is equal in capacity to one-half of each of the reagent holding chambers 40, 50, and 60. The quantity determining chamber 30 is 1.5 μl in capacity, while each of the reagent holding chambers 40, 50, and 60 is 3.0 μl in capacity.

The measuring chamber 70 has the shape of circle which is 2 mm in diameter in a sectional view taken by a plane perpendicular to a thickness direction of the disc 10 for determining a total cholesterol value, and about 1 μl in capacity. Considering that the depth of the measuring chamber 70 corresponds to an optical path, it is preferable that, in this embodiment, the measuring chamber 70 be 200 μm in depth. The detection of a chemical change resulting from a chemical reaction of reagent with cholesterol in one's fluid sample is performed through a step of detecting the change of absorbance of pigment contained in the reagent layer 5b in specific frequency. Therefore, a section defined as a ceiling of the measuring chamber 305 and a section defined as a bottom of the measuring chamber 305 have respective flat surfaces. Those sections corresponding to the measuring chamber 305 are transmissive within an optical range and the like including a wavelength of 650 nm.

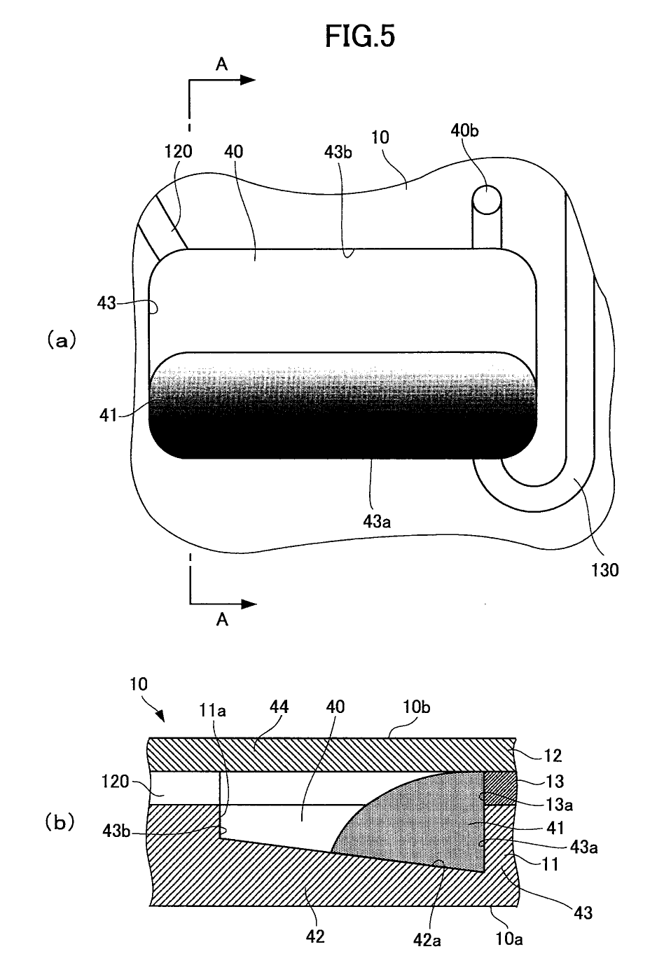

As shown in FIG. 5, the disc 10 for determining a total cholesterol value has a surface 42 defined as a reagent attaching surface to which the ChE layer 41 has been attached, a surface 43 defined as a surrounding surface extending from the surface 42, and a surface 44 facing the surface 42, and extending from the surface 43. The surfaces 42, 43, and 44 collectively form a reagent holding chamber 40. The reagent attaching surface 43b having a section 42a inclined with respect to one disc surface and connected to the surrounding surface, the surrounding surface having two sections facing each other, the solid reagent being attached to one section, and not attached to the other section. The reagent attaching surface 43b has a reagent attaching surface 43a connected to the section 42a inclined with respect to one disc surface, the ChE layer being attached to the reagent attaching surface 43a, and a section 43b which the ChE layer is not attached to. Surfaces forming the reagent holding chambers 50 and 60 are the same in construction as those of the reagent holding chamber 40.

Considering chemical reactions of solid reagents with a fluid sample and time required for determination of a total cholesterol value or the like, it is preferable to reduce the numbers of chambers in which the solid reagents are respectively held. In the disc 10 for determining a total cholesterol value, the ChE layer 41, the ChDH layer 51, and the WST-9 layer 61 are respectively held in the reagent holding chambers 40, 50, and 60 on the basis of following reasons.

The pH buffering agent is essential to adjust the hydrogen-ion exponent of the solution to an optimum hydrogen-ion exponent, or to alkalize the fluid sample. However the stability of ChDH is reduced in the alkaline solution. Therefore, the ChE layer 41 and the ChDH layer 51 are respectively held in the reagent holding chambers 40 and 50 in the disc 10 for determining a total cholesterol value. The pH buffering agent is contained in the ChE layer 41. As ChE, enzyme may be selected from among marketed products on the basis of the stability and the reactive property of the enzyme.

WST-9 has a tendency to inhibit a catalytic activity of ChDH. Therefore, the ChDH layer 51 and the WST-9 layer 61 are held in the respective reagent holding chambers 50 and 60 in the disc 10 for determining a total cholesterol value.

As shown in FIG. 4, the disc 10 for determining a total cholesterol value of a blood sample has a fluid passageway 110 connected to the blood cell component separating chamber 20, and connected to a section of the quantity determining chamber 30, the section being close to a center which the disc 10 for determining a total cholesterol value is rotated around (hereinafter simply referred to as “rotation center of disc”), in comparison with the remaining part of the quantity determining chamber 30, a fluid passageway 120 connected to a section of the quantity determining chamber 30, the section being far from the rotation center of the disc 10 in comparison with the remaining part of the quantity determining chamber 30, and connected to a section of the reagent holding chamber 40, the section being close to the rotation center of the disc 10 in comparison with the remaining part of the reagent holding chamber 40, a fluid passageway 130 connected to a section of the reagent holding chamber 40, the section being far from the rotation center of the disc 10 in comparison with the remaining part of the reagent holding chamber 40, and connected to a section of the reagent holding chamber 50, the section being close to the rotation center of the disc 10 in comparison with the remaining part of the reagent holding chamber 50, a fluid passageway 140 connected to a section of the reagent holding chamber 50, the section being far from the rotation center of the disc 10 in comparison with the remaining part of the reagent holding chamber 50, and connected to a section of the reagent holding chamber 60, the section being close to the rotation center of the disc 10 in comparison with the remaining part of the reagent holding chamber 60, a fluid passageway 150 connected to a section of the reagent holding chamber 60, the section being far from the rotation center of the disc 10 in comparison with the remaining part of the reagent holding chamber 60, and connected to a section of each of the measuring chamber 70 and the waste fluid chamber 80, the section being close to the rotation center of the disc 10 in comparison with the remaining part of the measuring chamber 70 or the waste fluid chamber 80, and a fluid passageway 160 connected to a section of the quantity determining chamber 30, the section being close to the rotation center of the disc 10 in comparison with the remaining part of the quantity determining chamber 30. Each of the fluid passageways 110 to 160 is 100 μm in depth defined in a thickness direction of the disc 10 for determining a total cholesterol value.

Here, the fluid passageway 110, 120, 130, 140, and 150 have curved sections close to the rotation center of the disc 10 for determining a total cholesterol value, in comparison with the remaining sections of the blood cell component separating chamber 20, the quantity determining chamber 30, and the reagent holding chambers 40, 50, and 60, respectively. The fluid passageway 150 has a curved section 151 and a section 152 located between the measuring chamber 70 and the waste fluid chamber 80, the section 152 being larger in diameter than the remaining part of the measuring chamber 70. The fluid passageway 160 has an air opening 160a to ensure that the fluid sample smoothly flows through the fluid passageway 160 toward the next chamber. In order to prevent the fluid sample from leaking from the disc 10 for analyzing a fluid sample through the air opening when the disc 10 for analyzing a fluid sample is rotated around the rotation center by the analyzing device 800, the air opening 160a is arranged at a special position.

The following description is directed to a method of producing a disc 10 for determining a total cholesterol value.

A polycarbonate plate 11 (see FIG. 5(b)) is molded with bores 11a corresponding to the blood cell component separating chamber 20, the quantity determining chamber 30, the reagent holding chambers 40, 50, and 60, the measuring chamber 70, and the waste fluid chamber 80, and bores corresponding to the inlet port 20a which the blood sample is introduced through, and the air openings 20b, 40b, 50b, 60b, and 160a.

A polyethylene terephthalate plate 13, which two-sided adhesive tape has added to, is formed with bores 13a (see FIG. 5(b)) corresponding to the blood cell component separating chamber 20, the quantity determining chamber 30, the reagent holding chambers 40, 50, and 60, the measuring chamber 70, the waste fluid chamber 80, and the passageways 110 to 160.

The polyethylene terephthalate plate 13 is then adhered well to the polycarbonate plate 11.

The ChE layer 41 is formed in the reagent holding chamber 40 through steps of placing 1.5 μl of aqueous solution on a section corresponding to the reagent holding chamber 40, and drying the aqueous solution in the reagent holding chamber 40 while placing the aqueous solution on the concave section under the force of gravity. Here, the aqueous solution consists of ChE, n-octyl-β-D-thioglucoside and sodium cholate which function as a surfactant to increase a catalytic activity of the ChE, Tris-hydrochloride which functions as pH buffering agent to adjust pH value of the fluid sample in a chemical reaction, and DI.

The ChDH layer 51 is formed in the reagent holding chamber 50 through steps of placing 1.5 μl of aqueous solution of ChDH and DI on a section corresponding to the reagent holding chamber 50, and drying the aqueous solution in the reagent holding chamber 50 while placing the aqueous solution on the concave section under the force of gravity.

The WST-9 layer 61 is formed in the reagent holding chamber 60 through steps of placing 1.5 μl of aqueous solution of WST-9 on a section corresponding to the reagent holding chamber 60, and drying the aqueous solution in the reagent holding chamber 60 while placing the aqueous solution on the concave section under the force of gravity.

The disc 10 for determining a total cholesterol value is produced through a step of having the polycarbonate plate 12 adhered well to the polyethylene terephthalate plate 13.

The following description is directed to the operation of the disc 10 for determining a total cholesterol value.

The blood sample is firstly introduced into the blood cell component separating chamber 20 through the inlet port 20a of the disc 10 for determining a total cholesterol value. The disc 10 for determining a total cholesterol value is mounted on an analyzing device 800 (see FIG. 1), and then rotated by the spindle motor 810 (see FIG. 1). The blood sample introduced into the blood cell component separating chamber 20 is separated by a centrifugal force into two components including a blood plasma component and a blood call component when the disc 10 is being rotated by the spindle motor 810. The surface of the blood plasma extracted from the blood sample in the blood cell component separating chamber 20 is balanced with the surface of the blood plasma flowed into the fluid passageway 110 under the condition that the disc 10 for determining a total cholesterol value is being rotated around its rotation axis. Even if the blood plasma flows into the fluid passageway 110, the blood plasma flowed into the fluid passageway 110 cannot reach the curved section 111 of the fluid passageway 110 by reason that the distance of the curved section 111 to the rotation axis is short in comparison with the distance of the blood cell component separating chamber 20 to the rotation axis.

When the disc 10 for determining a total cholesterol value is stopped by the spindle motor 810 (see FIG. 1), the blood plasma held in the blood cell component separating chamber 20 and the fluid passageway 110 flows by the capillary force toward the quantity determining chambers 30 through the fluid passageway 110. When the surface of the blood plasma reaches a section connected to the quantity determining chambers 30, the blood plasma stops without flowing into the quantity determining chambers 30 by the capillary force.

When the disc 10 for determining a total cholesterol value is rotated by the spindle motor 810, the blood plasma held in the fluid passageway 110 is introduced into the quantity determining chambers 30 in response to the centrifugal force. The fluid sample held in the blood cell component separating chamber 20 is siphoned and introduced into the quantity determining chambers 30 through the fluid passageway 110 by a siphon effect while the disc 10 for determining a total cholesterol value is rotated by the spindle motor 810. When the fluid sample introduced into the quantity determining chambers 30 is flowed to the fluid passageway 120, the fluid sample flowed to the fluid passageway 120 fails to reach a curved section 121 of the fluid passageway 120 by reason that the curved section 121 of the fluid passageway 120 is closer to the center of the disc 10 than the quantity determining chambers 30 is, and the centrifugal force keeps a balance between the fluid level of the fluid sample flowed to the fluid passageway 120 and the fluid level of the fluid sample introduced into the quantity determining chambers 30. When the fluid sample introduced into the quantity determining chambers 30 reaches a connected section of the fluid passageway 130, the fluid sample excessively introduced into the quantity determining chambers 30 is introduced into the waste fluid chamber 80 through the fluid passageway 160.

When the disc 10 for determining a total cholesterol value is stopped and then restarted by the spindle motor 810, the blood plasma flows into the reagent holding chamber 40 from the quantity determining chamber 30. The ChE layer 41 held in the reagent holding chamber 40 is solved, and reacts chemically, as shown by the chemical reaction formula (1), with the blood plasma flowed into the reagent holding chamber 40.

When the disc 10 for determining a total cholesterol value is stopped and then restarted by the spindle motor 810, the blood plasma flows into the reagent holding chamber 50 from the reagent holding chamber 40. The ChDH layer 51 held in the reagent holding chamber 50 is solved, and reacts chemically, as shown by the chemical reaction formula (2), with the blood plasma introduced into the reagent holding chamber 50.

When the disc 10 for determining a total cholesterol value is stopped and then restarted by the spindle motor 810, the blood plasma flows into the reagent holding chamber 60 from the reagent holding chamber 50. The WST-9 layer 61 held in the reagent holding chamber 60 is solved, and reacts chemically, as shown by the chemical reaction formula (3), with the blood plasma introduced into the reagent holding chamber 60.

When the disc 10 for determining a total cholesterol value is stopped by the spindle motor 810, the blood plasma flowed into the reagent holding chamber 60 and the fluid passageway 150 is siphoned by the capillary force of the fluid passageway 150 toward the measuring chamber 70 through the fluid passageway 150. When the fluid level of the blood plasma reaches a section 152 larger in diameter than the remaining section of the fluid passageway 150, the blood plasma is stopped by the section 152 without being siphoned by the capillary force of the fluid passageway 150.

When the disc 10 for determining a total cholesterol value is rotated by the spindle motor 810, the blood plasma flows into a large-diameter section 152 from the fluid passageway 150 in response to the centrifugal force, and then flows into the measuring chamber 70. As a result, the blood plasma in the reagent holding chamber 60 is siphoned by the capillary force of the fluid passageway 150, and flows into the measuring chamber 70 and the waste fluid chamber 80 through the fluid passageway 150 while the centrifugal force acts on the disc 10 for determining a total cholesterol value.

When the blood plasma flows into the measuring chamber 70, an optical pickup 212 (see FIG. 17) is moved in a radial direction by a transfer motor 21 (see FIG. 17) of the analyzing device. Then, the optical pickup 212 irradiates a light beam to the blood plasma in the measuring chamber 70, and detects a light from the blood plasma in the measuring chamber 70. The total cholesterol value is determined on the basis of the change of the reagent.

From the foregoing description, it will be understood that, in the disc 10 for determining a total cholesterol value, the ChE layer 41 can be uniformly formed in the reagent holding chamber 40, in comparison with the conventional device, on a section which has a tendency to have the solid reagent react chemically with the fluid sample flowed into the chamber when the ChE layer 41 is formed in the reagent holding chamber 40 through steps of placing an aqueous solution on a section corresponding to the reagent holding chamber 40, and drying the solution of ChE in the reagent holding chamber 40 while placing the solution of ChE on a section close to a surface 10a of the disc under the force of gravity.

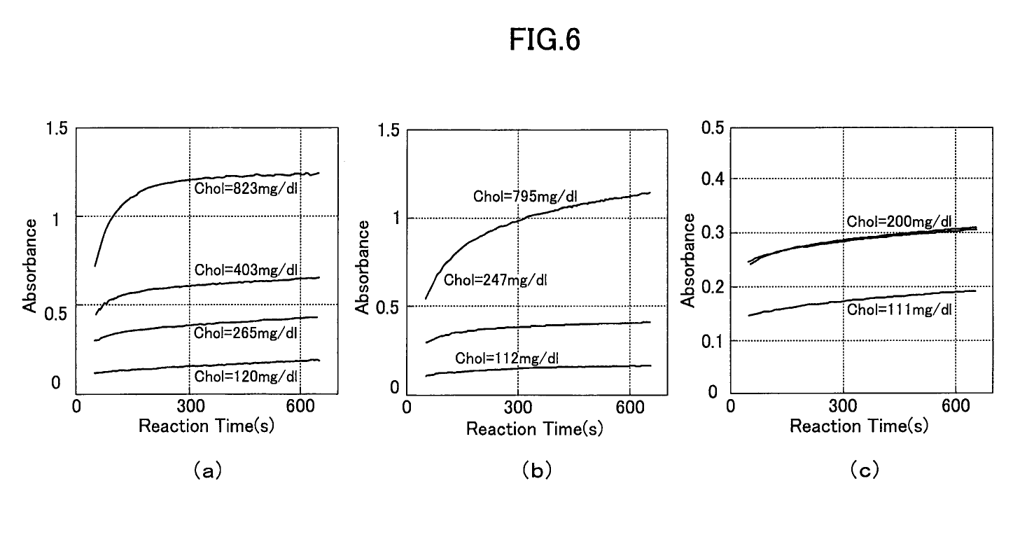

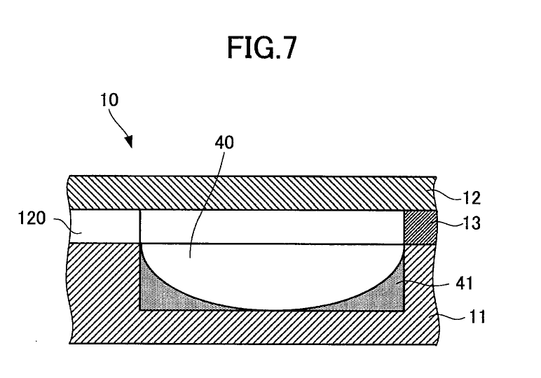

The ChE layer 41 is solved in the blood plasma through an agitation attributed to two factors, i.e., the flow of the blood plasma into the reagent holding chamber 40 and the diffusion of the reagent in the blood plasma in the reagent holding chamber 40. Therefore, the solution of the ChE layer 41 in the blood plasma flowing into the reagent holding chamber 40 extremely slows down if the ChE layer 41 is not uniform in thickness. It takes a long time to solve all the ChE layer 41 held in the reagent holding chamber 40 under the condition that the ChE layer 41 is partially thick. However, the disc 10 for determining a total cholesterol value can have ChE react quickly with the blood plasma, in comparison with the conventional device, by reason that the ChE layer 41 is uniformly formed in the reagent holding chamber 40 on a section which has a tendency to have the solid reagent react chemically with the blood plasma in the reagent holding chamber 40. Consequently, the disc 10 for determining a total cholesterol value can determine a total cholesterol value quickly. When, for example, the reagent holding chamber 40 has the shape shown in FIG. 5, the change of absorbance on the total cholesterol value of the blood plasma is shown in FIG. 6(a). When the reagent holding chamber 40 has the shape shown in FIG. 7, i.e., the same in shape as that of the conventional device, the change of absorbance on the total cholesterol value of the blood plasma is shown in FIG. 6(b). From FIG. 6, it will be understood that ChE reacts quickly with the blood plasma under the condition that the reagent holding chamber 40 has the shape shown in FIG. 5, when the total cholesterol value is relatively high, in comparison with the reagent holding chamber 40 has the shape shown in FIG. 7, i.e., the same in shape as that of the conventional device.

It is hard to expect an effect of an agitation attributed to the flow of the blood plasma into the reagent holding chamber 40 when the blood plasma is in the reagent holding chamber 40. Therefore, the concentration of ChE in the blood plasma is not equalized in every corner of the reagent holding chamber 40 if the ChE layer 41 held in the reagent holding chamber 40 is not uniform in thickness. However, the disc 10 for determining a total cholesterol value can determine the total cholesterol value quickly and accurately by having ChE deposited uniformly as the ChE layer 41 on a section which has a tendency to have the reagent react chemically with the blood plasma in the reagent holding chamber 40, and having the fluid sample solve quickly and uniformly, and react quickly and uniformly with ChE in the reagent holding chamber 40.

The surrounding surface 42 has a section 42a. The ChE layer 41 is formed in the reagent holding chamber 40 through a step of drying the solution of ChE in the reagent holding chamber 40 while placing the solution of ChE on a surface under the force of gravity. Therefore, the disc 10 for determining a total cholesterol value thus constructed according to the present invention can prevent the solution of ChE from reaching a section 43b which the reagent is not attached to, and prevent the ChE layer from reaching a section 43b which the reagent is not attached to.

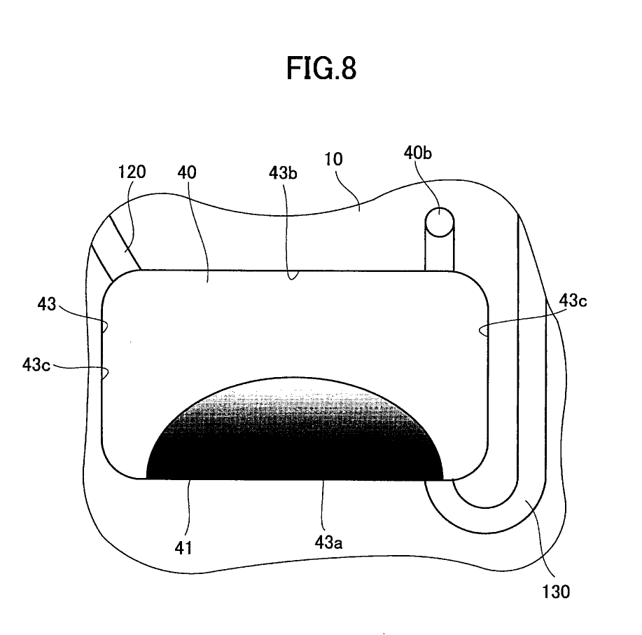

The ChE layer 41 shown in FIG. 8 can be formed in the reagent holding chamber 40 through a step of having the thickened solution of ChE attached to only a section 43a, and preventing the thickened solution of ChE from reaching a section 43b which the reagent is not attached to, and sections 43c which the reagent is not attached to. In the disc 10 for determining a total cholesterol value, the ChE layer 41 is not formed on a section 43c as shown in FIG. 8. As a result, the ChE layer 41 is solved quickly in the blood plasma.

The disc 10 for determining a total cholesterol value may have a section inclined a reagent attaching surface 43b having a section 42a inclined with respect to one disc surface and connected to the surrounding surface, the surrounding surface having two sections facing each other, the solid reagent being attached to one section, and not attached to the other section. Therefore, the disc 10 for determining a total cholesterol value thus constructed according to the present invention can prevent the solution of ChE from reaching a section 42b which the reagent is not attached to, and prevent the ChE layer from reaching a section 42b which the reagent is not attached to, even if the wetting characteristic of the surface to the solution of ChE is relatively high in the reagent holding chamber 40.

The reagent holding chambers 50 and 60 is the same in construction as the reagent holding chamber 40 described in the foregoing description.

If the reagent is deposited uniformly, and formed as a layer on a section which has a tendency to have the reagent react chemically with the fluid sample introduced into the chamber, a section, which the reagent is attached to, is not limited by the above-mentioned construction. In order to equalize the reagent layer to be formed on a section which has a tendency to have the solid reagent react chemically with the fluid sample in the chamber, the structure of the section may have a tendency to have the solid reagent react chemically with the fluid sample in the chamber. The structure of the section having a tendency to have the solid reagent react chemically with the fluid sample in the chamber will be more specifically described hereinafter.

### Third Embodiment

The third embodiment of the disc for analyzing a sample according to the present invention will be then described hereinafter with reference to accompanying drawings.

In order to provide an easy-to-understand explanation about the disc for analyzing a sample according to the present invention, the disc for analyzing a sample according to the present invention is used for determining a total cholesterol value from a blood plasma component of one's blood sample.

The reaction mechanism of the third embodiment is the same as that of the foregoing embodiment.

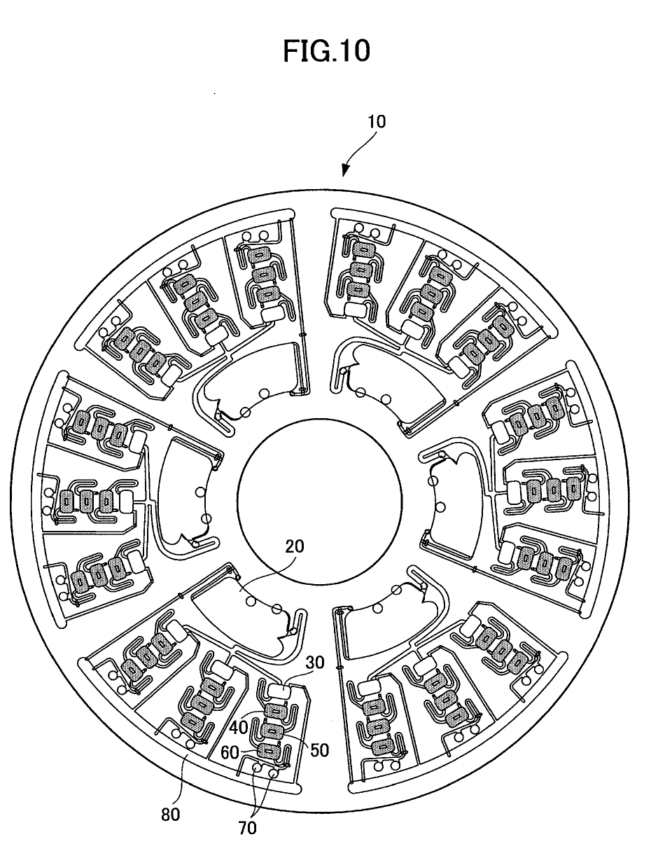

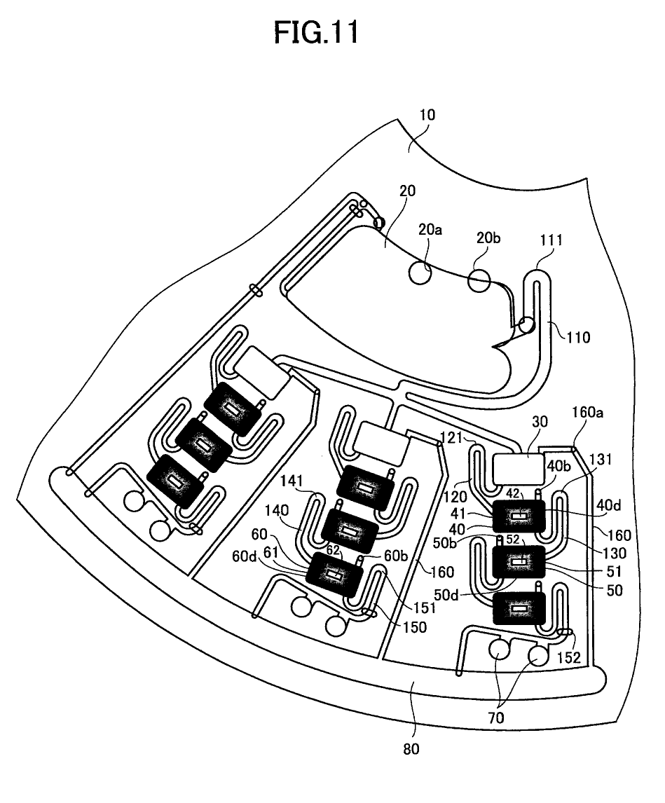

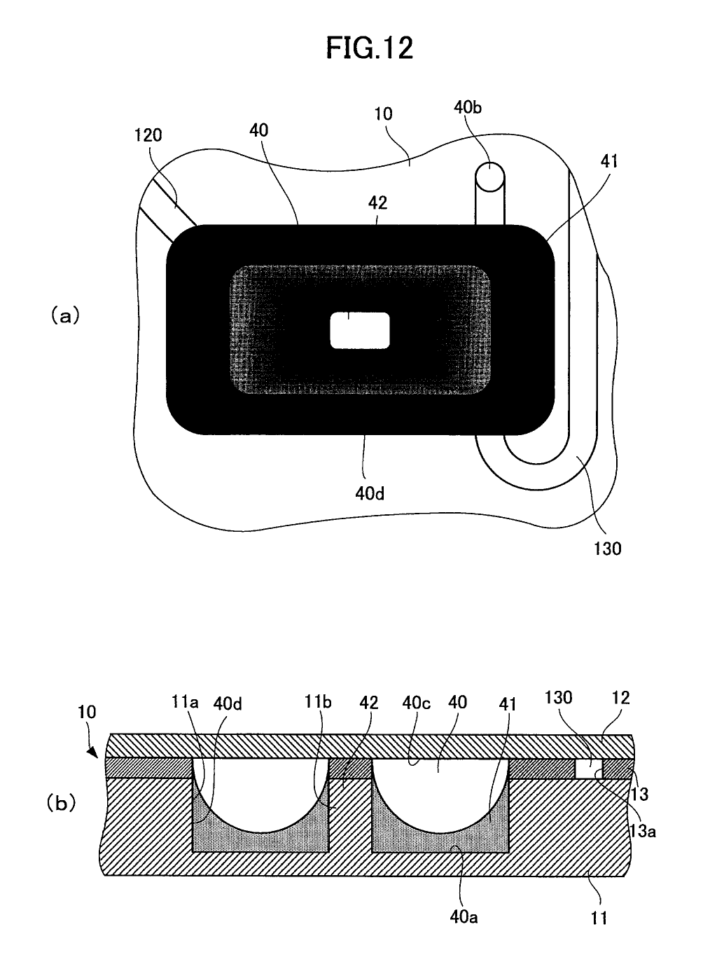

As shown in FIGS. 10 and 11, the disc 10 for determining a total cholesterol value has a blood cell component separating chamber 20 for eliminating, on the basis of a centrifugal separation method, a blood cell component from one's blood sample to extract a blood plasma component from the blood sample, a quantity determining chamber 30 for determining a predetermined quantity of blood plasma from the blood plasma extracted by the blood cell component separating chamber 20, a reagent holding chamber 40 having a ChE layer 41 formed as a solid reagent containing ChE on a surface 40a (see FIG. 12(b)) perpendicular to a thickness direction of the disc 10 for determining a total cholesterol value (hereinafter simply referred to as “thickness direction”), a reagent holding chamber 50 having a ChDH layer 51 formed as a solid reagent containing ChDH on a surface perpendicular to the thickness direction, a reagent holding chamber 60 having a WST-9 layer 61 formed as a solid reagent containing WST-9 on a surface perpendicular to a thickness direction, a measuring chamber 70 for measuring an absorbance of Formazan at a wavelength of 650 nm, and a waste fluid chamber 80 having a waste fluid stored therein. The chambers, the arrangement of the fluid passageways of the disc 10 according to the third embodiment are the same as those of the disc according to the foregoing embodiments.

The disc 10 for determining a total cholesterol value has a columnar section 42c projecting from a surface 40a of the reagent holding chamber 40, the columnar section 42c being connected to a surface 40c (see FIG. 12(b)) facing the surface 40a, and columnar sections 52 and 62 provided in the respective reagent holding chambers 42 and 62. As shown in FIG. 12(b), the ChE layer 41 deposited on a surface 40a is increased in thickness in the vicinity of the columnar section 42c and a surrounding surface 40d. The ChDH layer 51 disposed on a surface is increased in thickness in the vicinity of the columnar section 52 and a surrounding surface 50d. The WST-9 layer 61 deposited on a surface is increased in thickness in the vicinity of the columnar section 62 and a surrounding surface 60d.

From the viewpoint of their responsiveness and measuring time, it is preferable that the disc have one or two reagent holding chambers without increasing the number of reagent holding chambers. However, the disc 10 for determining a total cholesterol value has three reagent holding chambers 40, 50, and 60, which the ChE layer 41, the ChDH layer 51, and the WST-9 layer 61 are respectively held in, on the basis of reasons the same as that of the foregoing embodiments.

The following description is directed to a method of producing a disc 10 for determining a total cholesterol value.

A polycarbonate plate 11 (see FIG. 12(b)) is molded with bores 11a (see FIG. 12(b)) corresponding to the blood cell component separating chamber 20, the quantity determining chamber 30, the reagent holding chambers 40, 50, and 60, the measuring chamber 70, and the waste fluid chamber 80, projections 11b corresponding to the columnar sections 42c, 52, and 62 projecting from respective surfaces of the reagent holding chambers 40, 50, and 60, and bores corresponding to the inlet port 20a which the blood sample is introduced through, and the air openings 20b, 40b, 50b, 60b, and 160a.

A polyethylene terephthalate plate 13, which two-sided adhesive tape has added to, is formed with bores 13a (see FIG. 12(b)) corresponding to the blood cell component separating chamber 20, the quantity determining chamber 30, the reagent holding chambers 40, 50, and 60, the measuring chamber 70, the waste fluid chamber 80, and the passageways 110 to 160.

The polyethylene terephthalate plate 13 is then adhered well to the polycarbonate plate 11.

The ChE layer 41 is formed in the reagent holding chamber 40 through steps of placing 5 μl of aqueous solution on a section corresponding to the reagent holding chamber 40, and drying the aqueous solution. Here, the aqueous solution consists of ChE, n-octyl-P-D-thioglucoside and sodium cholate which function as a surfactant to increase a catalytic activity of the ChE, Tris-hydrochloride which functions as pH buffering agent to adjust pH value of the fluid sample in a chemical reaction, and DI. Additionally, the ChE layer 41 formed on the surface 40a is thick in the vicinity of the surrounding surface 40d and the columnar section 42 of the reagent holding chamber 40, and decreased with distance from the surrounding surface 40d and the columnar section of the reagent holding chamber 40, by reason that the aqueous solution is dried in the reagent holding chamber 40 while being affected by its own surface tension and the like.

The ChDH layer 51 is formed in the reagent holding chamber 50 through steps of placing 1.5 μl of aqueous solution of ChDH and DI on a section corresponding to the reagent holding chamber 50, and drying the aqueous solution in the reagent holding chamber 50 while placing the aqueous solution on the concave section under the force of gravity. Additionally, the ChDH layer 51 is thick in the vicinity of the surrounding surface 50d and the columnar section 52 of the reagent holding chamber 50, and decreased with distance from the surrounding surface 50d and the columnar section of the reagent holding chamber 50, by reason that the aqueous solution is dried in the reagent holding chamber 50 while being affected by its own surface tension and the like.

The WST-9 layer 61 is formed in the reagent holding chamber 60 through steps of placing 1.5 μl of aqueous solution on a section corresponding to the reagent holding chamber 60, and drying the aqueous solution in the reagent holding chamber 60 while placing the aqueous solution on the concave section under the force of gravity. Here, the aqueous solution consists of WST-9. Additionally, the WST-9 layer 61 is thick in the vicinity of the surrounding surface 60d and the columnar section 62 of the reagent holding chamber 60, and decreased with distance from the surrounding surface 60d and the columnar section of the reagent holding chamber 60, by reason that the aqueous solution is dried in the reagent holding chamber 60 while being affected by its own surface tension and the like.

The disc 10 for determining a total cholesterol value is produced through a step of having the polycarbonate plate 12 adhered well to the polyethylene terephthalate plate 13.

From the foregoing description, it will be understood that the ChE layer 41 can be uniformly formed in the reagent holding chamber 40, in comparison with the conventional device, on a section which has a tendency to have the solid reagent react chemically with the fluid sample flowed into the chamber, by reason that, when the ChE layer 41 is formed on a surface 40a of the reagent holding chamber 40 in step of drying the solution of ChE in the reagent holding chamber 40, ChE is thickly deposited on the surface 40a in the vicinity of not only the surrounding surface 40d and but also the columnar section 42 of the reagent holding chamber 40.

The ChE layer 41 is solved in the blood plasma through an agitation attributed to two factors, i.e., the flow of the blood plasma into the reagent holding chamber 40 and the diffusion of the reagent in the blood plasma in the reagent holding chamber 40. Therefore, the solution of the ChE layer 41 in the blood plasma flowing into the reagent holding chamber 40 extremely slows down if the ChE layer 41 is not uniform in thickness. It takes a long time to solve all the ChE layer 41 held in the reagent holding chamber 40 under the condition that the ChE layer 41 is partially thick. However, the disc 10 for determining a total cholesterol value can have ChE react quickly with the blood plasma, in comparison with the conventional device, by reason that the ChE layer 41 is uniformly formed in the reagent holding chamber 40 on a section which has a tendency to have the solid reagent react chemically with the blood plasma in the reagent holding chamber 40. Consequently, the disc 10 for determining a total cholesterol value can determine a total cholesterol value quickly.

It is hard to expect an effect of an agitation attributed to the flow of the blood plasma into the reagent holding chamber 40 when the blood plasma is in the reagent holding chamber 40. Therefore, the concentration of ChE in the blood plasma is not equalized in every corner of the reagent holding chamber 40 if the ChE layer 41 held in the reagent holding chamber 40 is not uniform in thickness. However, the disc 10 for determining a total cholesterol value can determine the total cholesterol value quickly and accurately by having ChE deposited uniformly as the ChE layer 41 on a section which has a tendency to have the reagent react chemically with the blood plasma in the reagent holding chamber 40, and having the fluid sample solve quickly and uniformly, and react quickly and uniformly with ChE in the reagent holding chamber 40.

The disc 10 for determining a total cholesterol value can allow the blood plasma to react with the ChE layer 41 quickly in comparison with the conventional device by reason that the blood plasma is agitated by the columnar section 42c of the reagent holding chamber 40 when the blood plasma flows into the reagent holding chamber 40. The disc 10 for determining a total cholesterol value can allow the blood plasma to quickly and uniformly react with the ChE layer 41 in comparison with the conventional device, and can quickly and accurately determine a total cholesterol value of a blood plasma component extracted from one's blood sample, by reason that the blood plasma is agitated by the columnar section 42c of the reagent holding chamber 40 when the blood plasma flows into the reagent holding chamber 40.

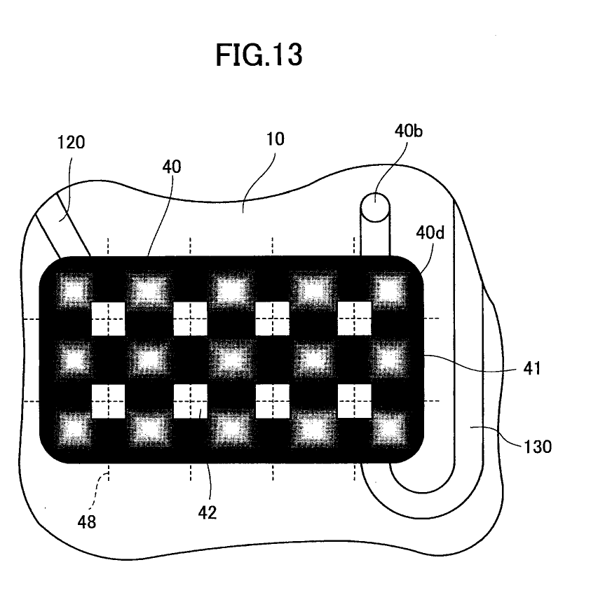

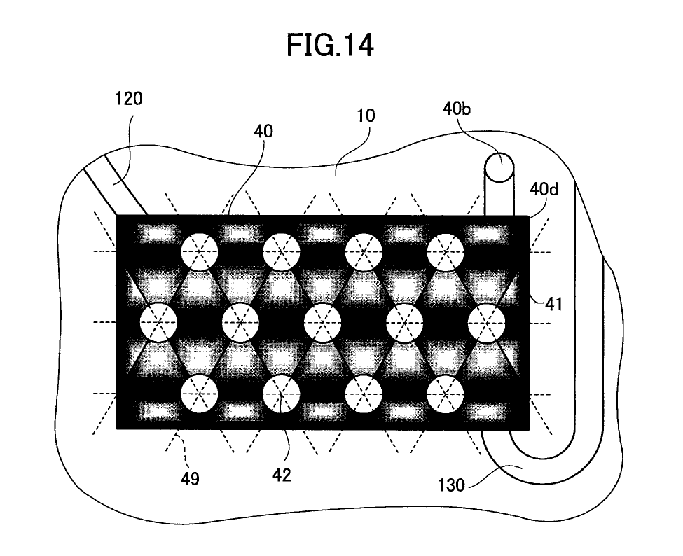

Additionally, the disc 10 for determining a total cholesterol value can have the ChE layer uniformed in thickness on a section which has a tendency to have the solid reagent react chemically with the fluid sample in the reagent holding chamber 40 by increasing the number of the columnar sections 42c per unit area on the surface 40a of the reagent holding chamber 40, and allow the blood plasma to be effectively agitated by the columnar section 42c of the reagent holding chamber 40 when the blood plasma flows into the reagent holding chamber 40. In the disc 10 for determining a total cholesterol value and having a number of columnar sections 42c arranged at regular intervals, ChE can be uniformly deposited as a layer on a section which has a tendency to have the solid reagent react chemically with the fluid sample in the chamber, in comparison with a disc having a number of columnar sections 42c arranged at irregular intervals. As shown in FIG. 13, the columnar sections 42c may be located at intersecting points of an orthogonal grid 48 drawn imaginarily on the surface 40a (see FIG. 12(b)) of the reagent holding chamber 40 in the disc 10 for determining a total cholesterol value. The fluid sample can be more effectively agitated by the columnar sections under the condition that columnar sections are closely located at regular intervals. For example, the columnar sections 42c may be located at intersecting points of an equilateral triangle grid 49 drawn imaginarily on the surface 40a of the reagent holding chamber 40 in the disc 10 for determining a total cholesterol value as shown in FIG. 14. More specifically, columnar sections 42c may be equidistant from one columnar section 42c located at a center of a circle, and equiangularly located on a circumferential line of the circle. For example, six columnar sections 42c may be equidistant from one columnar section 42c located at a center of a circle, and equiangularly located on a circumferential line of the circle. Even if seven or more columnar sections 42 equidistant from one columnar section 42c located at a center of a circle, and equiangularly located on a circumferential line of the circle, the columnar sections 42c cannot be arranged at regular intervals in every corner of the chamber. Therefore, it is preferred that six columnar sections 42c is equidistant from one columnar section 42c located at a center of a circle, and equiangularly located on a circumferential line of the circle as shown in FIG. 14.

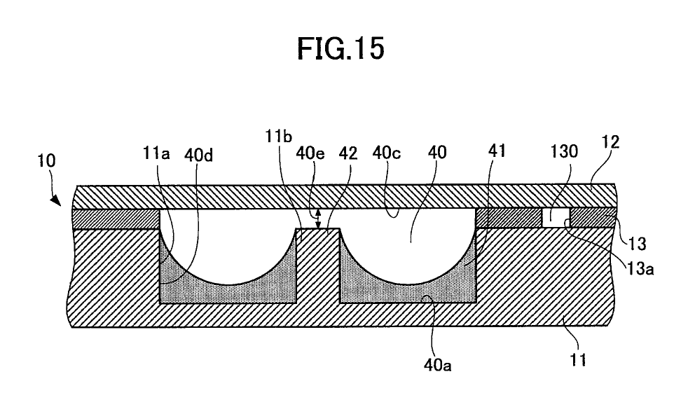

The disc 10 for determining a total cholesterol value can agitate the blood plasma strongly by using the columnar section 42c when the blood plasma flows into the reagent holding chamber 40, by reason that the disc 10 for determining a total cholesterol value has a columnar section 42c projected from a surface 40a to a surface 40c facing the surface 40a and connected to the surface 40c as shown in FIG. 12(b), in comparison with a disc having a columnar section 42c projected from a surface 40a toward a surface 40c facing the surface 40a and not connected to the surface 40c as shown in FIG. 15. However, it is not necessary to have the columnar section 42c connected to the surface 40c as shown in FIG. 15. The disc 10 for determining a total cholesterol value can have the blood plasma flow quickly into the reagent holding chamber 40 through a gap between the columnar section 42c and the surface 40c, by reason that the disc 10 for determining a total cholesterol value has a columnar section 42c not connected to the surface 40c, in comparison with a disc having a columnar section 42c connected to the surface 40c as shown in FIG. 12(b).

Each of the columnar section 52 of the reagent holding chamber 50 and the columnar section 62 of the reagent holding chamber 60 is the same in construction as the above-mentioned columnar section 42c of the reagent holding chamber 40, and will not be described hereinafter.

In this embodiment, the determination of a total cholesterol value in blood plasma is performed through a step of detecting the change of absorbance of WST-9. The determination of a total cholesterol value in blood plasma may be performed on the basis of another method. The reagent layer may contain potassium ferricyanide in place of WST-9. When the reagent layer is solved in blood plasma or the like, the potassium ferricyanide contained in the reagent layer is reduced to ferricyanide ion in blood plasma. The disc 10 for determining a total cholesterol value may have at least two electrodes provided in the measuring chamber 70. One functions as a counter electrode, while the other functions as a working electrode. The analyzing device has terminals to be connected to the electrodes provided in the measuring chamber 70. The determination of a total cholesterol value in blood plasma may be performed through a step of detecting an oxidation current, when ferrocyanide ion reduced through oxidization of cholesterol in blood plasma is again oxidized, by using electrodes to which a voltage is applied. In this case, redox compound may be contained in the reagent in place of potassium ferricyanide in order to have electron transfer between redox compound and NADH.

The disc for analyzing a sample according to the present invention is not limited by the analysis of a total cholesterol value in blood plasma explained in this embodiment, and may be applied to an analysis of another component of any sample through an optical change or an electrochemical change of the component.

In this embodiment, the blood plasma introduced into the chamber is transferred by the combination of the centrifugal force and the capillary force. However, the blood plasma introduced into the chamber may be transferred on the basis of another method. As an example, the blood plasma introduced into the chamber may be transferred by gravity force or may be transferred by a pump. Each fluid passageway intervening between two chambers, or an upper end section of an upstream chamber and a lower end section of a downstream chamber can be linearly arranged along a direction of the centrifugal force produced and controlled by the rotation of the disc. The disc for analyzing a sample according to the present invention can be small in size by reason that an area occupied by fluid passageways thus constructed as previously mentioned can be reduced. In order to transfer the fluid sample in stages through steps of holding the fluid sample in an upstream chamber, allow the fluid sample to flow into a fluid passageway and move the fluid sample to the next chamber, holding the fluid sample in that, and move the fluid sample to further chamber in response to the centrifugal force produced by the rotation of the disc, it is important to control the centrifugal force acting on the disc, the capillary force of the fluid passageway, the surface tension of the fluid sample at a connection point of the chamber and the fluid passageway or on an inner surface of the fluid passageway, a boundary length of the connection point and the fluid passageway, a distance from a connection point between the chamber and the fluid passageway to the fluid level of the fluid sample held in the chamber, a holding of the fluid sample in the chamber under the condition that the centrifugal force is acting on the disc, and a transfer of the fluid sample to the next chamber through the fluid passageway. In order to transfer, in stages, the fluid sample to chambers connected in series, it is important that an upper limit of the centrifugal force acting on a chamber close to the rotation center of the disc be smaller than an upper limit of the centrifugal force acting on a chamber distant from the rotation center of the disc. If the upper limit of the centrifugal force acting on a chamber close to the rotation center of the disc exceeds the upper limit of the centrifugal force acting on a chamber distant from the rotation center of the disc, the fluid sample flow the chambers in series without stopping at the next chamber. Therefore, it is preferable to reduce the number of the chambers connected in series.

In this embodiment, the disc has the shape of circle, its rotation center being defined in an inner section surrounded by a peripheral section of the disc. As shown in FIG. 10, a plurality of sample analyzers may be formed in a disc. The analyzing device has a turntable directly connected to a rotation axis of the spindle motor 810. The turntable has a section for retaining the disc. For example, the turntable may have a depressed section operable to retain the disc. The disc retained by the turntable may be rotated around a rotation center distant from or defined outside the disc. In this case, the turntable may have a section formed with a through bore or the like, and related to the measuring chamber 70.

While there has been described in the foregoing embodiments about four structural features including: (1) a structural feature characterized in that the surfaces includes a surface, which the solid reagent is attached to, having the shape of concave, (2) a structural feature characterized in that the surfaces includes two surfaces facing each other, one surface, which the solid reagent is attached to, having the shape of concave, the other surface having a section having the shape of convex, (3) a structural feature characterized in that the surfaces includes a reagent attaching surface which the solid reagent is attached to, an opposing surface facing the reagent attaching surface, and a surrounding surface extending from the reagent attaching surface to the opposing surface, the reagent attaching surface having a section inclined with respect to one disc surface and connected to the surrounding surface, the surrounding surface having two sections facing each other, the solid reagent being attached to one section, and not attached to the other section, (4) a structural feature characterized by a columnar section projecting from reagent attaching surface which the solid reagent is attached to, the disc for analyzing a sample according to the present invention is not limited by the above-mentioned structural features, and may have one or more structural features to equalize the reagent layer to be formed on a section which has a tendency to have the reagent react chemically with the fluid sample in the reagent holding chamber.

### INDUSTRIAL APPLICABILITY OF THE PRESENT INVENTION

As will be seen from the foregoing description, it will be understood that the disc for analyzing a sample according to the present invention has an advantageous effect of analyzing the sample quickly and accurately by having the fluid sample react quickly and uniformly with the solid reagent held in the chamber, and is useful as a disc to be used in a blood sample analyzing device and the like, by reason that the solid reagent is uniformly formed on and attached as a layer to a section which has a tendency to have the solid reagent react chemically with the fluid sample introduced into the chamber.

1. A disc for analyzing a fluid sample, said disc having surfaces forming a chamber which said fluid sample is introduced into, and a solid reagent held in said chamber, said solid reagent being solved in said fluid sample introduced into said chamber, wherein
said disc has at least one of four structural features including:
(1) a structural feature characterized in that said surfaces includes a surface, which said solid reagent is attached to, having the shape of concave,
(2) a structural feature characterized in that said surfaces includes two surfaces facing each other, one surface, which said solid reagent is attached to, having the shape of concave, the other surface having a section having the shape of convex,
(3) a structural feature characterized in that said surfaces includes a reagent attaching surface which said solid reagent is attached to, an opposing surface facing said reagent attaching surface, and a surrounding surface extending from said reagent attaching surface to said opposing surface, said reagent attaching surface having a section inclined with respect to one disc surface and connected to said surrounding surface, said surrounding surface having two sections facing each other, said solid reagent being attached to one section, and not attached to the other section,
(4) a structural feature characterized by a columnar section projecting from reagent attaching surface which said solid reagent is attached to, and connected to an opposing surface facing said reagent attaching surface,
said solid reagent held in said chamber is uniformly deposited on a section which has a tendency to have said solid reagent react chemically with said fluid sample introduced into said chamber.

2. A disc for analyzing a fluid sample according to claim 1, wherein said chamber is defined by a gap formed with at least two opening, and defined by a spacer intervening between an upper substrate and a lower substrate, said upper substrate having a convex section, said lower substrate having a concave section, said reagent being attached to said concave section of said lower substrate.

3. A disc for analyzing a fluid sample according to claim 2, wherein said convex section of said upper substrate and said concave section of said lower substrate are similar in shape to each other.

4. A disc for analyzing a fluid sample according to claim 2, wherein a distance between said convex section of said upper substrate and said concave section of said lower substrate is substantially fixed in every corner of said convex section and said concave section.

5. A disc for analyzing a fluid sample according to claim 2, wherein said convex section has a curved surface, and said concave section has a curved surface.

6. A disc for analyzing a fluid sample according to claim 2, which further has two or more chambers connected through one or more fluid passageways, each of said chambers being the same in construction as said chamber.

7. A disc for analyzing a fluid sample according to claim 1, wherein said reagent attaching surface has a section other than said section inclined with respect to one surface of said disc, and connected to a section which said reagent is not attached, and which forms part of said surrounding surface.

8. A method of producing a disc defined in claim 1, said disc having surfaces forming a chamber, and a solid reagent held in said chamber and attached to one of said surfaces, said solid reagent being solved in said fluid sample when said fluid sample is introduced into said chamber, wherein
said surfaces includes a reagent attaching surface located on one side of said disc, said solid reagent being attached to said reagent attaching surface, and a surrounding surface extending from said reagent attaching surface, said reagent attaching surface having a section inclined with respect to said one side of said disc, and connected to said surrounding surface, said surrounding surface having two sections facing each other, said solid reagent being attached to one of said sections, and not attached to the other of said sections, and
said solid reagent is deposited on said reagent attaching surface through a step of drying a solution of said solid reagent.

9. A disc for analyzing a fluid sample according to claim 1, wherein said disc has a chamber formed therein, said disc having a columnar section disposed in said chamber and projected from a surface of said chamber, a solid reagent attached to one surface of said chamber being solved in said fluid sample introduced into said chamber.

10. A disc for analyzing a fluid sample according to claim 9, wherein said disc has two or more columnar sections located at intersecting points of an orthogonal grid.

11. A disc for analyzing a fluid sample according to claim 9, wherein said disc has two or more columnar sections located at intersecting points of an orthogonal grid.

12. A disc for analyzing a fluid sample according to claim 9, wherein said columnar section extends from one of said surfaces, and is connected to the other of said surfaces.

13. A disc for analyzing a fluid sample according to claim 9, wherein said columnar section extends from one of the surfaces toward the other of said surfaces.

14. A method of producing a disc for analyzing a fluid sample introduced into a chamber, according to claim 9, wherein
said disc has a columnar section disposed in said chamber, and projected from a surface of said chamber, and a solid reagent attached to said surface of said chamber, said solid reagent being solved in said fluid sample introduced into said chamber,
said method having a step of having said solid reagent attached to said surface of said chamber by drying a fluid reagent introduced into said chamber.
